SEQUENCE LISTING (34 sequences)

SEQ ID NO: 1 - DNA, 1317 bp, Clostridium phytofermentans
atgaagaactatttccccaacgtcccagaagtcaaatacgaaggtccaaactccacaaatcctttcgcttttaaatattatgatgctaataaagtagtcgccggtaagaccatgaaggagcattgtagattcgctctatcctggtggcacactttgtgtgccggtggtgctgatccattcggagtaactactatggacaggacctacggtaacattaccgacccaatggaactagctaaggccaaagttgatgctggtttcgaactgatgactaagctgggcatcgagttcttctgcttccatgatgccgacattgctccagaaggtgacaccttcgaagagtccaagaagaatctgttcgagattgttgattacatcaaggagaagatggaccaaaccggcatcaagttgttatggggcactgctaacaactttagtcaccccaggttcatgcacggtgcatcaacttcttgtaatgccgatgttttcgcttatgctgctgcgaaaataaagaacgctttagatgcgaccatcaagttgggcggtaagggttatgtcttttggggtggtagagaaggttacgagaccctgctgaatactgacctgggcttagaactggacaacatggctaggctaatgaagatggccgtagaatacggtagggctaatggattcgacggtgacttctacatcgagcctaaacccaaggaacctactaagcaccagtacgacttcgacactgctaccgtattagcttttttaaggaagtacgggttggaaaaagacttcaagatgaacatcgaagccaatcacgccacactagcaggccacacattcgagcatgagttagctatggctagggtaaacggtgcattcggttctgttgatgctaaccaaggtgacccaaacttaggatgggacacggatcaattccccacagacgttcattctgctactcttgctatgctggaggtcttgaaagccggtggtttcacaaatggcggcctgaactttgatgcgaaagttcgtaggggttcattcgagtttgacgatattgcctatggttacattgctggtatggatactttcgcgttagggttaattaaagctgctgaaatcattgatgacggtagaattgccaagtttgtggatgacaggtatgcctcttacaagaccggtattggtaaagcgatcgttgacggaactacctctttggaagaattggaacaatacgtgttgactcattctgaacctgtcatgcaatctggtagacaagaggttctggaaactattgtcaacaacatattgtttagataa

SEQ ID NO: 2 - protein, 438 aa, Clostridium phytofermentans
MKNYFPNVPEVKYEGPNSTNPFAFKYYDANKVVAGKTMKEHCRFALSWWHTLCAGGADPFGVTTMDRTYGNITDPMELAKAKVDAGFELMTKLGIEFFCFHDADIAPEGDTFEESKKNLFEIVDYIKEKMDQTGIKLLWGTANNFSHPRFMHGASTSCNADVFAYAAAKIKNALDATIKLGGKGYVFWGGREGYETLLNTDLGLELDNMARLMKMAVEYGRANGFDGDFYIEPKPKEPTKHQYDFDTATVLAFLRKYGLEKDFKMNIEANHATLAGHTFEHELAMARVNGAFGSVDANQGDPNLGWDTDQFPTDVHSATLAMLEVLKAGGFTNGGLNFDAKVRRGSFEFDDIAYGYIAGMDTFALGLIKAAEIIDDGRIAKFVDDRYASYKTGIGKAIVDGTTSLEELEQYVLTHSEPVMQSGRQEVLETIVNNILFR

SEQ ID NO: 3 - DNA, 1326 bp, Abiotrophia defective
atgagtgaattgttccaaaacatcccaaaaatcaaatacgaaggtgcaaattccaaaaatcctttggcttttcattattatgatgctgaaaaaatagtcctcggtaagaccatgaaggagcatttgccattcgctatggcatggtggcacaatttgtgtgccgctggtactgatatgttcggacgtgatactgcggacaagtcctttggtttggaaaaaggctcaatggaacatgctaaggccaaagttgatgctggtttcgaatttatggaaaagctgggcattaaatacttctgcttccatgatgtagaccttgttccagaagcttgcgacattaaagagaccaattctcgactggacgaaatttctgattacatcttggagaagatgaagggcactgatattaagtgtttatggggcactgctaatatgttttctaaccccaggttcgtgaacggtgcaggatctactaatagtgccgatgtttactgttttgctgctgcgcaaataaagaaagcattagatattaccgtcaagttgggcggtagaggttatgtcttttggggtggtagagaaggttacgagaccctgctgaatactgacgtgaaatttgaacaggaaaacattgctaatctaatgaagatggccgtagaatacggtaggtctattggattcaaaggtgacttctacatcgagcctaaacccaaggaacctatgaagcaccagtacgacttcgacgctgctaccgcaataggttttttaaggcagtacgggttggataaagacttcaaattgaacatcgaagccaatcacgccacactagcaggacactcattccagcatgagttacgtatttctagtattaacggtatgttgggttctgttgatgctaaccaaggtgacatgttgttaggatgggacacggatgaatttccctttgacgtttatgatactactatgtgtatgtatgaggtccttaaaaacggtggtttgacaggcggctttaactttgatgcgaaaaatcgtaggccttcatacacgtatgaagatatgttctatggtttcattcttggtatggattctttcgcgttagggttgataaaagctgctaaattgattgaagaaggtacacttgacaattttattaaggaaaggtataaatcttttgaatccgaaattggtaaaaaaattagatccaaatcagcctctttgcaagaattggcagcttatgctgaggaaatgggtgctcccgcgatgccgggttcaggtaggcaagagtatctgcaagctgctctcaaccaaaatttgtttggtgaagtgtaataa

SEQ ID NO: 4 - protein, 440 aa, Abiotrophia defective
MSELFQNIPKIKYEGANSKNPLAFHYYDAEKIVLGKTMKEHLPFAMAWWHNLCAAGTDMFGRDTADKSFGLEKGSMEHAKAKVDAGFEFMEKLGIKYFCFHDVDLVPEACDIKETNSRLDEISDYILEKMKGTDIKCLWGTANMFSNPRFVNGAGSTNSADVYCFAAAQIKKALDITVKLGGRGYVFWGGREGYETLLNTDVKFEQENIANLMKMAVEYGRSIGFKGDFYIEPKPKEPMKHQYDFDAATAIGFLRQYGLDKDFKLNIEANHATLAGHSFQHELRISSINGMLGSVDANQGDMLLGWDTDEFPFDVYDTTMCMYEVLKNGGLTGGFNFDAKNRRPSYTYEDMFYGFILGMDSFALGLIKAAKLIEEGTLDNFIKERYKSFESEIGKKIRSKSASLQELAAYAEEMGAPAMPGSGRQEYLQAALNQNLFGEV

SEQ ID NO: 5 - DNA, 1317 bp, Ruminococcus flavifaciens
atggaatttttctccaacatcggaaaaatccaataccaaggtccaaaatccacagatcctttgtcttttaaatattataatcctgaagaagtaatcaacggtaagaccatgagggagcatttgaaattcgctctatcctggtggcacactatgggtggcgatggtactgatatgttcggatgtggtactacggacaagacctggggtcaatccgacccagcggcaagagctaaggccaaagttgatgctgctttcgaaattatggataagctgagcattgattactactgcttccatgatagagacctttctccagaatatggctccttgaaagcgaccaatgatcaactggacattgttactgattacatcaaggagaagcagggcgataaattcaagtgtttatggggcactgctaaatgctttgatcaccccaggttcatgcacggtgcaggaacttctcctagtgccgatgttttcgctttttctgctgcgcaaataaagaaagcattagaatctaccgtcaagttgggcggtaatggttatgtcttttggggtggtagagaaggttacgagaccctgctgaatactaacatgggcttagaactggacaacatggctaggctaatgaagatggccgtagaatacggtaggtctattggattcaaaggtgacttctacatcgagcctaaacccaaggaacctactaagcaccagtacgacttcgacactgctaccgtattaggttttttaaggaagtacgggttggataaagacttcaagatgaacatcgaagccaatcacgccacactagcacaacacacattccagcatgagttacgtgtggctagggataacggtgtattcggttctattgatgctaaccaaggtgacgtattgttaggatgggacacggatcaattccccacaaacatttatgatactactatgtgtatgtatgaggtcattaaagccggtggtttcacaaatggcggcctgaactttgatgcgaaagctcgtaggggttcattcacgcctgaagatattttctatagttacattgctggtatggatgctttcgcgttagggtttagagcagctcttaaattgattgaagacggtagaattgacaagtttgtggctgacaggtatgcctcttggaataccggtattggtgcagatattattgccggaaaagccgattttgcatcattggaaaaatatgctttggaaaaaggtgaagttaccgcgtcattgtcttctggtagacaagagatgctggaatctattgtcaacaacgtattgtttagtttgtaa

SEQ ID NO: 6 - protein, 438 aa, Ruminococcus flavifaciens
MEFFSNIGKIQYQGPKSTDPLSFKYYNPEEVINGKTMREHLKFALSWWHTMGGDGTDMFGCGTTDKTWGQSDPAARAKAKVDAAFEIMDKLSIDYYCFHDRDLSPEYGSLKATNDQLDIVTDYIKEKQGDKFKCLWGTAKCFDHPRFMHGAGTSPSADVFAFSAAQIKKALESTVKLGGNGYVFWGGREGYETLLNTNMGLELDNMARLMKMAVEYGRSIGFKGDFYIEPKPKEPTKHQYDFDTATVLGFLRKYGLDKDFKMNIEANHATLAQHTFQHELRVARDNGVFGSIDANQGDVLLGWDTDQFPTNIYDTTMCMYEVIKAGGFTNGGLNFDAKARRGSFTPEDIFYSYIAGMDAFALGFRAALKLIEDGRIDKFVADRYASWNTGIGADIIAGKADFASLEKYALEKGEVTASLSSGRQEMLESIVNNVLFSL

SEQ ID NO: 7 - DNA, 1323 bp, Ruminococcus flavifaciens
atggaatttttcaagaacatctctaagataccatacgaaggcaaagactctaccaatccattagcattcaagtactacaatcctgacgaagtaatcgacggtaagaagatgagagacatcatgaagtttgctttgtcttggtggcatactatgggaggtgatggtactgatatgtttggctgtggtactgctgataagacatggggcgagaatgatccagctgctagagctaaagctaaagttgatgccgcatttgaaatcatgcagaagttatccattgattacttctgcttccatgatagagatttgtctccagagtacggttctttgaaggacacaaacgctcaattggacattgtcactgactacatcaaggctaaacaagctgaaaccggtttgaaatgtctttggggtactgctaagtgcttcgaccatccaagattcatgcacggtgctggtacttctccttcagcggatgtcttcgcattctcagctgctcaaatcaagaaagctctggaatctaccgtcaagttgggtggaactggttatgtcttctggggtggtagagaaggatatgaaacgttgttgaatactaacatgggacttgaattggacaacatggctaggttgatgaagatggccgttgagtatggtaggtctattggtttcaaaggtgacttctacattgaacctaagccaaaggaaccaactaagcatcaatacgactttgacactgctacagtcttgggctttctgagaaagtacggcctggacaaagacttcaagatgaacatagaagccaatcatgcaactttagcgcaacataccttccagcacgaattgtgtgtcgccagaactaatggtgctttcggttctattgatgctaatcaaggtgatcccttgttgggttgggatacagatcagtttcctacaaacatctatgatactactatgtgcatgtacgaagttatcaaagctggtggtttcactaatggtggtcttaactttgatgctaaagctagaagaggttctttcactccagaagatattttctattcttacattgctggtatggatgctttcgctttaggttacaaagctgcttctaagctaatcgctgatggtaggattgatagcttcattagcgatagatatgcttcttggtctgaaggtattggtttggacatcatttccggcaaagctgatatggcggctttagagaagtatgctttggagaaaggagaggtcactgattctatctcttctggaagacaggaactgttagagtccattgttaacaacgtaatcttcaacctataataa

SEQ ID NO: 8 - protein, 439 aa, Ruminococcus flavifaciens
MEFFKNISKIPYEGKDSTNPLAFKYYNPDEVIDGKKMRDIMKFALSWWHTMGGDGTDMFGCGTADKTWGENDPAARAKAKVDAAFEIMQKLSIDYFCFHDRDLSPEYGSLKDTNAQLDIVTDYIKAKQAETGLKCLWGTAKCFDHPRFMHGAGTSPSADVFAFSAAQIKKALESTVKLGGTGYVFWGGREGYETLLNTNMGLELDNMARLMKMAVEYGRSIGFKGDFYIEPKPKEPTKHQYDFDTATVLGFLRKYGLDKDFKMNIEANHATLAQHTFQHELCVARTNGAFGSIDANQGDPLLGWDTDQFPTNIYDTTMCMYEVIKAGGFTNGGLNFDAKARRGSFTPEDIFYSYIAGMDAFALGYKAASKLIADGRIDSFISDRYASWSEGIGLDIISGKADMAALEKYALEKGEVTDSISSGRQELLESIVNNVIFNL

SEQ ID NO: 9 - DNA, 1410 bp, Phytopthara infestans
atgcaacatcaagtgaaagaatatttcccaaacgtcccaaaaatcacattcgaaggtcaaaatgccaaaagtgttttggcttatcgtgaatataatgcttcagaagtaatcatgggtaagaccatggaggagtggtgtagattcgctgtgtgttattggcacacttttggtaactctggttctgatccgttcggaggtgaaacttataccaatagattgtggaatgaatcattggaaagagctaatatttcttctagggaaagattgttggaagctgctaagtgcaaagctgatgctgctttcgaaacttttacaaagctgggcgttaaatactacaccttccatgatgtagaccttatttcagaaggtgccaaccttgaagagtcccaatctctactggacgaaatttctgattacttgttggataagcagaatcaaactggtgttaggtgtttatggggcactactaatttgtttggtcacagaaggttcatgaacggtgcatcaactaatcctgatatgaaagttttcgctcatgctgctgcgagagtaaagaaagcaatggaaattaccttgaagttgggcggtcaaaattttgtcttttggggtggtagagaaggtttccagtccattctgaatactgacatgaaaactgaactggatcacatggctgctttttttaagttggtcgtagcatacaaaaaggaacttggagccacatttcaattcttggtcgagcctaaacccagggaacctatgaagcaccagtacgactacgacgctgctaccgtagtagcttttttacatacgtacgggttgcaaaatgacttcaaattgaacatcgaacccaatcacaccacactagcaggacacgattacgagcatgatatatattatgctgctagttacaaaatgttgggttctgttgattgtaacacaggtgacccgttggtaggatgggacacggatcaatttttgatggacgaaaaaaaagctgttttggttatgaaaaagatcgttgaaatcggtggtttggcaccaggcggcttgaactttgatgcgaaagttcgtagggaatcaaccgatttggaagatattttcattgctcacattggtagtatggattgtttcgcgagagggttgagacaagctgctaaattgcttgaaaaaaatgaacttggcgaattggttaagcaaaggtatgcatcttggaaatccacacttggtgaaagaattgaacaaggacaagccactttggaagaagtggcagcttatgctaaggaaagtggtgaacccgatcatgtgtcaggtaagcaagagttggcggaacttatgtggagcacagttgcgttggctacagggatttggcaagatcatgttacttgttctttgactaaaaattggtgttaa

SEQ ID NO: 10 - protein, 469 aa, Phytopthara infestans
MQHQVKEYFPNVPKITFEGQNAKSVLAYREYNASEVIMGKTMEEWCRFAVCYWHTFGNSGSDPFGGETYTNRLWNESLERANISSRERLLEAAKCKADAAFETFTKLGVKYYTFHDVDLISEGANLEESQSLLDEISDYLLDKQNQTGVRCLWGTTNLFGHRRFMNGASTNPDMKVFAHAAARVKKAMEITLKLGGQNFVFWGGREGFQSILNTDMKTELDHMAAFFKLVVAYKKELGATFQFLVEPKPREPMKHQYDYDAATVVAFLHTYGLQNDFKLNIEPNHTTLAGHDYEHDIYYAASYKMLGSVDCNTGDPLVGWDTDQFLMDEKKAVLVMKKIVEIGGLAPGGLNFDAKVRRESTDLEDIFIAHIGSMDCFARGLRQAAKLLEKNELGELVKQRYASWKSTLGERIEQGQATLEEVAAYAKESGEPDHVSGKQELAELMWSTVALATGIWQDHVTCSLTKNWC

SEQ ID NO: 11 - DNA, 54 bp, Artificial Sequence
  Description of Artificial Sequence Synthetic oligonucleotide
gctcattagaaagaaagcatagcaatctaatctaagttttggatcccaaacaaa

SEQ ID NO: 12 - DNA, 54 bp, Artificial Sequence
  Description of Artificial Sequence Synthetic oligonucleotide
acttgataatgaaaactataaatcgtaaagacataagagatccgccatatgtta

SEQ ID NO: 13 - DNA, 54 bp, Artificial Sequence; identical to SEQ ID NO: 11
  Description of Artificial Sequence Synthetic primer

SEQ ID NO: 14 - DNA, 54 bp, Artificial Sequence; identical to SEQ ID NO: 12
  Description of Artificial Sequence Synthetic primer

SEQ ID NO: 15 - DNA, 26 bp, Artificial Sequence
  Description of Artificial Sequence Synthetic primer
acggtcttcaatttctcaagtttcag

SEQ ID NO: 16 - DNA, 28 bp, Artificial Sequence
  Description of Artificial Sequence Synthetic primer
tcgtactggtgcttagtaggttccttgg

SEQ ID NO: 17 - DNA, 26 bp, Artificial Sequence
  Description of Artificial Sequence Synthetic primer
acggtcttcaatttctcaagtttcag

SEQ ID NO: 18 - DNA, 24 bp, Artificial Sequence
  Description of Artificial Sequence Synthetic primer
gcgaaagaatccataccaagaatg

SEQ ID NO: 19 - DNA, 27 bp, Artificial Sequence
  Description of Artificial Sequence Synthetic primer
ttatgtcttttggggtggtagagaagg

SEQ ID NO: 20 - DNA, 25 bp, Artificial Sequence
  Description of Artificial Sequence Synthetic primer
tattcgtgaaacttcgaacactgtc

SEQ ID NO: 21 - DNA, 24 bp, Artificial Sequence
  Description of Artificial Sequence Synthetic primer
tgtcttctggggtggtagagaagg

SEQ ID NO: 22 - DNA, 25 bp, Artificial Sequence
  Description of Artificial Sequence Synthetic primer
tattcgtgaaacttcgaacactgtc

SEQ ID NO: 23 - DNA, 24 bp, Artificial Sequence
  Description of Artificial Sequence Synthetic primer
tgtcttttggggtggtagagaagg

SEQ ID NO: 24 - DNA, 24 bp, Artificial Sequence
  Description of Artificial Sequence Synthetic primer
gcgaaacaatccatactaccaatg

SEQ ID NO: 25 - DNA, 27 bp, Artificial Sequence
  Description of Artificial Sequence Synthetic primer
ttatgtcttttggggtggtagagaagg

SEQ ID NO: 26 - DNA, 25 bp, Artificial Sequence
  Description of Artificial Sequence Synthetic primer
tattcgtgaaacttcgaacactgtc

SEQ ID NO: 27 - DNA, 27 bp, Artificial Sequence
  Description of Artificial Sequence Synthetic primer
ttatgtcttttggggtggtagagaagg

SEQ ID NO: 28 - DNA, 25 bp, Artificial Sequence
  Description of Artificial Sequence Synthetic primer
tattcgtgaaacttcgaacactgtc

SEQ ID NO: 29 - DNA, 26 bp, Artificial Sequence
  Description of Artificial Sequence Synthetic primer
acggtcttcaatttctcaagtttcag

SEQ ID NO: 30 - DNA, 24 bp, Artificial Sequence
  Description of Artificial Sequence Synthetic primer
gcgaaagcatccataccagcaatg

SEQ ID NO: 31 - DNA, 26 bp, Artificial Sequence
  Description of Artificial Sequence Synthetic primer
acggtcttcaatttctcaagtttcag

SEQ ID NO: 32 - DNA, 24 bp, Artificial Sequence
  Description of Artificial Sequence Synthetic primer
gcgaaagcatccataccagcaatg

SEQ ID NO: 33 - DNA, 24 bp, Artificial Sequence
  Description of Artificial Sequence Synthetic primer
tgtcttttggggtggtagagaagg

SEQ ID NO: 34 - DNA, 24 bp, Artificial Sequence
  Description of Artificial Sequence Synthetic primer
gcgaaacaatccatactaccaatg

(19) United States
(12) Patent Application Publication
(10) Pub. No.: US 2015/0050658 A1
(43) Pub. Date: Feb. 19, 2015

(54) GENE SHUFFLING METHODS

(71) Applicant: Codexis, Inc., Redwood City, CA (US)

(72) Inventor: Catherine M. Cho, Redwood City, CA (US)

(73) Assignee: Codexix, Inc., Redwood City, CA (US)

(21) Appl. No.: 14/385,060

(22) PCT Filed: Mar. 12, 2013
(86) PCT No.: PCT/US2013/030526

§ 371 Date: Sep. 12, 2014

Related U.S. Application Data
(60) Provisional application No. 61/611,484, filed on Mar. 15, 2012.

(51) Int. Cl. C12N 15/10

(52) U.S. Cl. CPC C12N 15/1027
USPC 435/6.12; 435/91.5

(57) ABSTRACT

Disclosed methods pertain to nucleic acid shuffling techniques that employ repeated short extension cycles. In each such cycle, strand extension along a template fragment is limited such that the strand extends only for a relatively short length (e.g., a few base pairs). Repeated short extension cycles cause many template switches during shuffling and thereby produce chimeric products with many crossovers. The methods may employ a pre-shuffling truncation or excision operation in which one or more parent nucleic acids has a portion of its full-length sequence truncated or excised. Shuffling with truncated parent nucleic acids introduces crossovers at the location of the truncation. Apparatus for implementing the disclosed methods may include appropriately configured thermocycling tools.

### CROSS REFERENCE TO RELATED APPLICATIONS

This application claims benefit of U.S. Provisional Patent Application No. 61/611,484, filed Mar. 15, 2012, which application is incorporated herein by reference in its entirety and for all purposes.

### SEQUENCE LISTING

The instant application contains a Sequence Listing which has been submitted in ASCII format via EFS-Web and is hereby incorporated by reference in its entirety. Said ASCII copy, created on Mar. 11, 2013, is named CDXSP016WO_SL.txt and is 34,300 bytes in size.

### BACKGROUND

Various methods are used to identify polypeptides having desired activities such as therapeutic effects, the ability to produce useful compositions from feed stocks, etc. Directed evolution and other protein engineering technologies can be used to discover or enhance the activity of polypeptides of commercial interest. For example, if the activity of a known enzyme is insufficient for a commercial process, directed evolution may be used to improve the enzyme's activity on a substrate of interest.

Current methods of directed evolution are often limited by the time and cost required to identify useful polypeptides. In some instances, it may take months or years, at great expense, to find a single such polypeptide, if one is ever found. Part of the problem arises from the great number of polypeptide variants that must be screened. Another part of the problem arises from limited exploration of sequence-activity space afforded by existing techniques. Thus, there is a need for improved methods that identify novel polypeptide variants having a desired activity.

### SUMMARY

Various methods for efficiently introducing diversity and exploring sequence space are described here. Libraries produced directly from these methods contain high fractions of protein variants harboring cross-overs between two or more parental genes. The methods produce these variants efficiently without the need for extensive screening to remove frame-shift mutants.

Disclosed methods pertain to nucleic acid shuffling techniques that employ repeated short extension recombination cycles. In each such cycle, strand extension along a template fragment is limited such that the strand extends only for a relatively short length (e.g., a few base pairs). Repeated short extension cycles cause many template switches during shuffling and thereby produce chimeric products with many crossovers. The methods may employ a pre-shuffling truncation or excision operation in which one or more parent nucleic acids has a portion of its full-length sequence truncated or excised. Shuffling with truncated parent nucleic acids introduces crossovers at the location of the truncation. Apparatus for implementing the disclosed methods may include appropriately configured thermal cycling tools.

In one aspect, this disclosure pertains to methods of conducting nucleic acid recombination to facilitate incorporation of crossovers in variant sequences. Such methods may be characterized by the following operations: (a) combining fragments of two or more parent nucleic acids; (b) annealing single stranded fragments from the two or more parent nucleic acids to produce annealed single stranded fragments; (c) incompletely extending the annealed single stranded fragments to produce incompletely extended single stranded fragments; (d) denaturing the incompletely extended single stranded fragments produced in (c); and (e) repeating (b)-(d) at least about 5 times to produce variant sequences. In some embodiments, operations (b)-(d) are repeated at least about 10 times, or at least about 15 times. The repetitions of (b) comprise annealing the incompletely extended single stranded fragments from (c).

At least some of the annealed single stranded fragments from (b) have overhanging single stranded portions attached to a double stranded portion. In certain embodiments, on average across the annealed fragments from the two or more parent nucleic acids, the extension performed in (c) covers not more than about 50% of the length of the overhanging single stranded portion of the annealed single stranded fragments existing prior to extension.

The combining in (a) may be performed with single stranded or double stranded fragments. Additionally, operation (a) need not be performed in all embodiments. For example, the two or more parent nucleic acids may be fragmented while they are present as a mixture in a single medium.

The above process may include a further operation (f) of identifying one or more recombinant proteins encoded by one or more variant sequences from (e), where the one or more recombinant proteins have at least one beneficial property. In one example, the at least one of the recombinant proteins is an enzyme such as a cellulase, reductase, transferase, transaminase, isomerase, protease, oxidase, kinase, synthase, or esterase. The recombinant proteins identified in (f) may be used for various purposes. For example, they may be used to generate a sequence activity model by the following steps: (i) assaying and sequencing the one or more recombinant proteins; and (ii) developing a sequence activity model from assay and sequence information for the recombinant proteins.

The parent nucleic acids may originate from various sources. For example, at least one of the parent nucleic acids may be a wild type nucleic acid sequence. In certain embodiments, the two or more parent nucleic acids have sequences with between about 50 and about 85 percent sequence identity.

The parent nucleic acids may be subjected to various treatments before or during the operations set forth above. For example, the method may include an additional operation of truncating a region of at least one of the two or more parent nucleic acids to produce a truncated fragment. In such cases, the method may optionally be performed in a manner is which at least one of the two or more parent nucleic acids is not truncated at a region corresponding the region truncated in the at least one parent nucleic acid.

Fragments of the parent nucleic acids may be produced according to various methods. For example, the fragments may be produced by endonuclease cleaving. The fragments may also be produced by cleavage at positions comprising uracil in the parent nucleic acids. In some cases, the fragments are produced by a method that does not include polymerase extension on a template comprising an unfragmented full-length parent nucleic acid. In some embodiments, the fragments are not produced by a method in which fragments are produced by extensions from external primers. In some cases, some of the fragments are produced by chemical synthesis.

In certain embodiments, operation (c) involves incompletely extending the annealed single stranded fragments by not more than about 35% of the overhanging single stranded portion, on average. In some examples, the incompletely extending operation may involve exposing the annealed single stranded fragments to polymerase and nucleotide triphosphates at a temperature of between about 58° C. and about 75° C. for a duration of between about 5 seconds and about 20 seconds.

In a specific embodiment, (i) the annealing in (b) is conducted at a temperature of between about 38° C. and about 50° C.; (ii) the extending in (c) is conducted at a temperature of between about 58° C. and about 75° C. for a duration of about 10 seconds to about 18 seconds; and (iii) the denaturing in (d) is conducted at a temperature of between about 80° C. and about 160° C. for a duration of about 10 seconds to about 50 seconds.

In some methods, the incompletely extension in (c) comprises a self-priming reaction in a medium that does not contain external primers. In further examples, the incompletely extension in (c) is performed in a medium that does not contain unfragmented full-length parent nucleic acids.

In certain embodiments, the above method may include additional operations to assemble the variant sequences produced in (e). In one example, the assembly process may be characterized by the following operations, preformed after (e): (f) repeating the annealing of single stranded fragments as in (b); (g) extending the annealed single stranded fragments to produce extended single stranded fragments; (h) denaturing the extended single stranded fragments produced in (g); and (i) repeating (f)-(h) at least about 10 times. The distance covered by the extending performed in (g) is significantly greater than that of the extensions in (c), on average across the annealed fragments from the two or more parent nucleic acids, the extension. In some embodiments, extending the single stranded fragments in (g) is conducted at a temperature of between about 58° C. and about 75° C. for a duration of about 18 seconds to about 60 seconds. In some embodiments, the annealing temperature is gradually increased during successive repetition recited in (i).

Another aspect of the disclosure pertains to methods of conducting nucleic acid recombination using the following operations: (a) truncating a region of at least one of two or more parent nucleic acid to produce at least one truncated parent nucleic acid; (b) fragmenting and combining the at least one truncated parent nucleic acid of (a) and at least one other parent nucleic acid that is not truncated in a region corresponding to the region truncated in the at least one truncated parent nucleic acid; (c) annealing single stranded fragments from the two or more parent nucleic acids to produce annealed single stranded fragments, where at least some of the annealed single stranded fragments have overhanging single stranded portions attached to a double stranded portion; (d) incompletely extending the annealed single stranded fragments, to produce incompletely extended single stranded fragments; (e) denaturing the incompletely extended single stranded fragments produced in (d); and (f) repeating (c)-(e) to produce variant sequences. The repetitions of (c) involve annealing the incompletely extended single stranded fragments from (d). Additionally, the extension in (d) is, on average across the annealed fragments from the two or more parent nucleic acids, not more than about 50% of the overhanging single stranded portion of the annealed single stranded fragments.

The truncating in (a) can remove a subsequence of the parent nucleic acid at any position over the full-length of the parent. For example, operation (a) may involve truncating a parent nucleic acid by removing a segment encoding a nitrogen terminal region of a protein encoded by the parent nucleic acid and truncating another parent nucleic acid by removing a segment encoding a carbon terminal region of a protein encoded by the other parent nucleic acid.

In certain embodiments, the truncating is performed by amplifying a parent nucleic acid in the presence of at least one primer complementary to an internal sequence of the at least one parent nucleic acid to produce at least one amplified parent nucleic acid. In certain embodiments, the amplifying comprises incorporating uracil nucleotides in the amplicons of at least one amplified parent nucleic acid. In some embodiments, the fragmenting comprises cleaving the amplicons at the uracil containing positions of the amplified parent nucleic acids.

Various options described above with respect to the incomplete extension, annealing, and denaturing operations may be applied to this aspect of the invention as well. Further, this aspect may include additional operations to assemble the variant sequences produced in (f).

In certain embodiments, the extending in (d) comprises incompletely extending the annealed single stranded fragments by not more than about 25% of the overhanging single stranded portion, on average.

Yet another aspect of the disclosure concerns additional methods of conducting nucleic acid recombination. This aspect may be characterized by the following operations: (a) truncating a region of at least one of two or more parent nucleic acid to produce at least one truncated parent nucleic acid; (b) fragmenting and combining the at least one truncated parent nucleic acid of (a) and at least one other parent nucleic acid that is not truncated in a region corresponding to the region truncated in the at least one truncated parent nucleic acid; (c) annealing single stranded fragments from the two or more parent nucleic acids to produce annealed single stranded fragments; (d) extending the annealed single stranded fragments to produce extended single stranded fragments; (e) denaturing the extended single stranded fragments produced in (d); and (f) repeating (c)-(e) at least about 5 times to produce variant sequences. The repetitions of (c) involve annealing the extended single stranded fragments from (d). Additionally, at least some of the annealed single stranded fragments in (c) have overhanging single stranded portions attached to a double stranded portion.

In some embodiments, the methods include an additional operation of aligning the parent nucleic acids to identify one or more regions of homology. In further embodiments, the methods may involve creating a primer complementary to at least one identified region of homology. The primer may be used in (i) truncating the region of the at least one parent nucleic acid to produce the at least one truncated parent nucleic acid and/or (ii) recovering full-length nucleic acids from the variant sequences. The two or more parent nucleic acids used in the methods may have substantially the same length and have between about 50 and about 85% sequence identity.

Additionally, the truncating operation may involve truncating at least two of the two or more parent nucleic acids. In further embodiments, the truncating operation comprises truncating at least one of the two or more parent nucleic acids by removing a segment encoding a nitrogen terminal region of a protein encoded by the at least one parent nucleic acid and truncating at least one other of the parent nucleic acids by removing a segment encoding a carbon terminal region of a protein encoded by the other parent nucleic acid.

In some embodiments, the truncating comprises amplifying the at least one parent nucleic acid in the presence of at least one primer complementary to an internal sequence of the at least one parent nucleic acid to produce at least one amplified parent nucleic acid. In one example, the amplifying comprises incorporating uracil nucleotides in the amplicons of at least one amplified parent nucleic acid, and then cleaving the amplicons at the uracil containing positions of the amplified parent nucleic acids.

In certain embodiments, the fragmenting in (b) comprises a process that does not include polymerase extension on a template comprising an unfragmented full-length or truncated parent nucleic acid. In certain embodiments, the fragmenting comprises a process in which fragments are not produced by extensions from primers. The fragments combined in (b) may be combined in non-equimolar amounts or in substantially equimolar amounts.

In some embodiments, the methods of this aspect further include an operation of extending the variant sequences to produce nucleic acids having substantially the same length as at least one of the parent nucleic acids. The extending may involve amplifying the variant sequences with flanking primers complementary to the terminal regions of at least one of the parent nucleic acids.

In one example, the extending in (d) comprises a self-priming reaction in a medium that does not contain external primers. Additionally, the extending in (d) may be performed in a medium that does not contain unfragmented full-length parent nucleic acids.

The other operations recited in this aspect may be performed in accordance with the variations set forth above for the other aspects. Further, the additional operations such as the assembly of variants, the choice parent nucleic acids, the producing sequence activity models from data about expressed variants, and the like may be performed as described above.

These and other features of the disclosed embodiments will be described in more detail below with reference to the associated drawings.

### BRIEF DESCRIPTION OF THE DRAWINGS

FIG. 1A is a schematic depiction of nucleic acid manipulations that take place in accordance with certain embodiments.

FIG. 1B is a schematic representation of a short extension procedure performed in accordance with certain embodiments.

FIG. 2 is a flow chart depicting an embodiment of the family shuffling procedures disclosed herein.

FIG. 3A is a schematic depiction of three different truncation/excision options.

FIG. 3B is a schematic depiction of three additional truncation/excision options.

FIG. 4 depicts results obtained using a shuffling procedure as disclosed in the example section provided herein.

### DETAILED DESCRIPTION

### I. Introduction and Overview

Family shuffling is one example of directed evolution. It is a technique that allows acceleration of in vitro evolution by combining diversity found in homologous genes. Typically, libraries of chimeric genes are generated by random fragmentation of a pool of related genes, followed by reassembly of the fragments in a self-priming polymerase reaction. Template switching—which is the hybridization of a single strand to multiple other single stands (templates) over the course of family shuffling—causes crossovers in areas of sequence homology. When sequence homologies are relatively low, reassembly of parental genes or chimeras with few crossovers are favored. [Jeff—Somewhere explain that chemical synthesis will work.]

For context and without limiting the scope or application of the methods disclosed herein, there are other techniques described in literature that are intended to create chimeric progeny of distantly related genes. Such techniques include ITCHY (Incremental truncation for the creation of hybrid enzyme: Nature Biotechnology 1999 (17), 1205-1209) and SCRATCHY (PNAS, 2001(98): 11248-11253). These truncation-based methods are intended to produce relatively large numbers of crossovers. However these methods suffer from certain disadvantages such as being limited to two parental genes and producing variant libraries in which a significant fraction of the variants contain frame-shifts due to insertions and deletions caused by random truncation and ligation. As a consequence, these methods require labor-intensive screening efforts to identify the relatively few in-frame variants present in a library.

The methods described herein involve shuffling of two or more parent genes or other parent nucleic acids. In certain embodiments, the parent nucleic acids have relatively low sequence similarity but are recombined in the disclosed shuffling methods. The disclosed methods generally ensure a low frequency of parent nucleic acids occurring in a resulting library. In certain embodiments described here, the number of nucleic acid chain extension cycles and the extension conditions are designed to produce chimeric genes with significant numbers of crossovers.

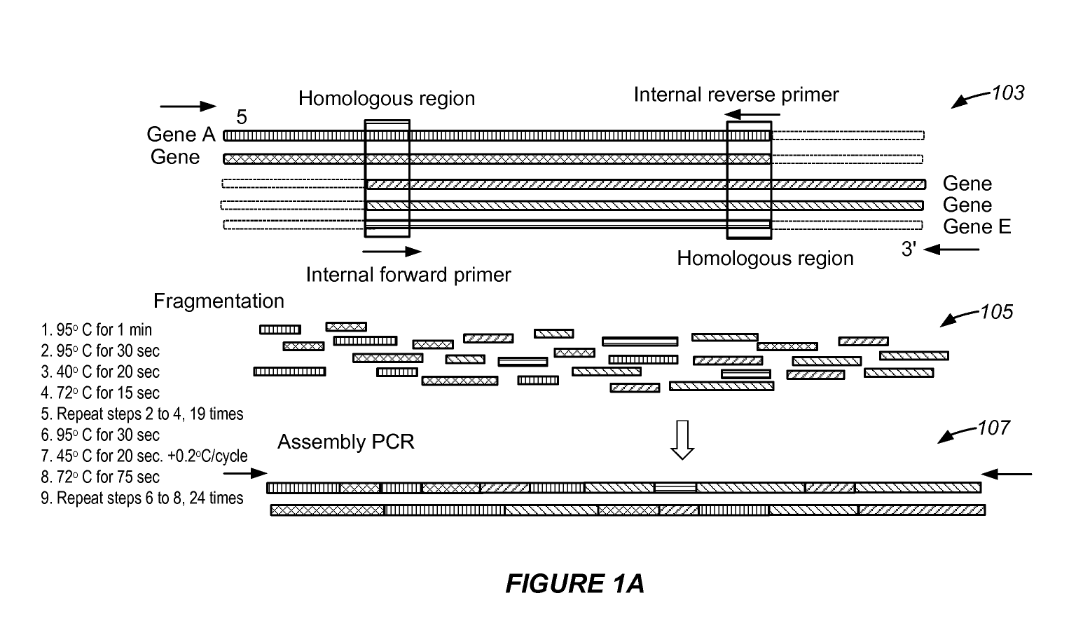

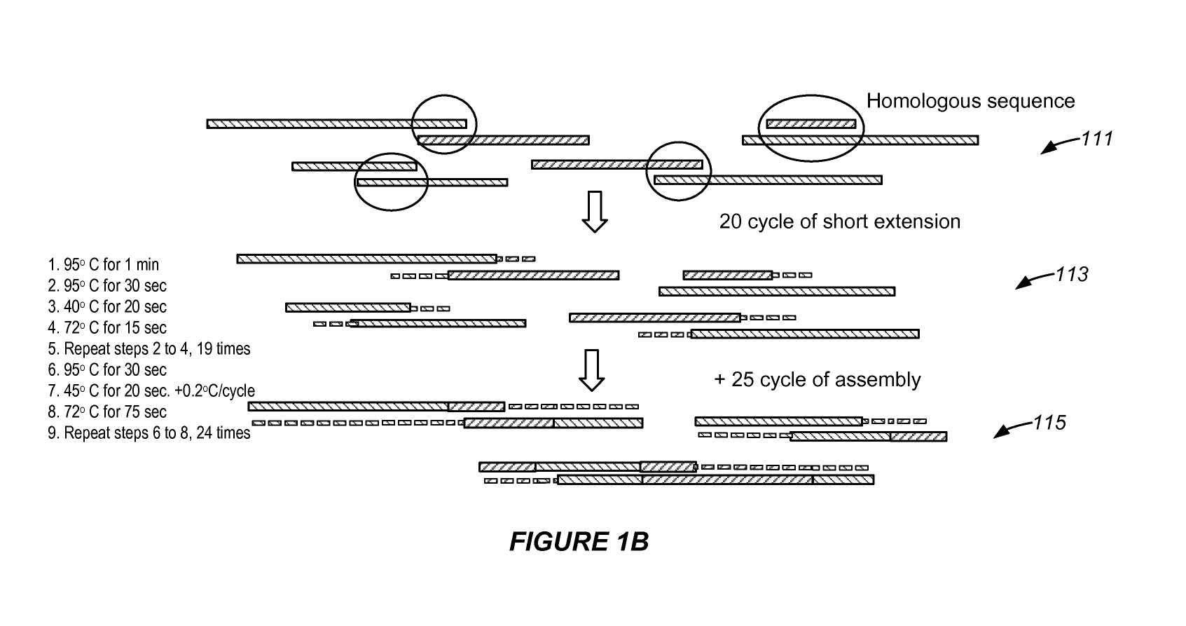

FIG. 1A presents an exemplary embodiment, in which parent nucleic acids are truncated as part of a shuffling procedure. In the depicted embodiment, the shuffling procedure employs five separate parent nucleic acids that are all approximately the same length. In this example, each of the parental genes is initially truncated at one or both of the terminal regions. The collection of truncated parent nucleic acids is schematically depicted in the Figure by reference number 103. The truncation may be accomplished by, for example, amplifying each of the parental genes with an internal primer complementary to an internal portion of the associated parent nucleic acid at the point of intended truncation. While all parent nucleic acids are shown to be truncated in this Figure, in some embodiments, not all of the parental nucleic acids are truncated. Indeed, in some embodiments, only one or a fraction of the parent nucleic acids is truncated.

In certain embodiments and consistent with the depicted embodiment of FIG. 1A, the five parent nucleic acids are fragmented and the fragments are then mixed and reassembled to produce multiple chimeric nucleic acids (e.g., chimeric genes). The schematic depiction of the fragmentation and assembly operations is shown by reference numerals 105 and 107 in FIG. 1A. In certain embodiments, full-length nucleic acids are rescued by conducting PCR (polymerase chain reaction) on the assembled fragments using flanking primers. This rescue procedure is described in more detail below.

In the embodiments illustrated in FIG. 1A, the truncation point in each of the parent nucleic acids occurs at a region of homology among some or all of the parent nucleic acids. This normally ensures a significant degree of recombination between fragments of different parent nucleic acids at the point of truncation. Consequently, a high fraction of the resulting chimeric nucleic acids have crossover points at the position of truncation. This result is depicted in the two chimeric nucleic acids shown in the products 107 of FIG. 1A. When the illustrated procedure is followed, few if any variants contain the full sequence of any of the parent nucleic acids (i.e., there is a very low level of parental background). Indeed, if all parent nucleic acids are truncated, there be little or no parental background found in the resulting chimeric nucleic acids. However, it should be understood that in certain embodiments, at least one parental sequence is not truncated. FIG. 1A illustrates an example in which all parent sequences are truncated, but this need not be the case.

In certain embodiments, the shuffling procedure includes a series of short extension cycles beginning with fragments from parent nucleic acids, and optionally including a truncation procedure such as that illustrated in FIG. 1A. Each of the short extension cycles extends a hybridized single-strand of nucleic acid by a relatively short distance, e.g., about 50% or less of the length of the overhanging strand of a complementary single-strand. A sufficient number of these short extension cycles are performed to ensure that many template switches occur during the shuffling. Optionally, after the short extension cycles are completed, one or more cycles of longer extension are performed. These longer extension cycles are referred to herein as “assembly cycles.”

FIG. 1B schematically depicts an example of a short extension shuffling assembly procedure encompassed by the present invention. In the depicted embodiment, two parent nucleic acids are fragmented and then mixed and exposed to hybridizing conditions to produce the hybridized pairs depicted at the process stage identified by reference numeral 111 of the figure. As shown, hybridization occurs between homologous sequences. In the depicted embodiment, 20 cycles of short extension polymerase chain reaction (PCR) are performed (See, reference numeral 113 of the Figure). Each of these cycles is performed under conditions that limit the extension to a relatively small fraction of the overhang from the complementary single-strand. For example, the duration of the extension portion of a cycle is relatively short to limit the number of bases that can be incorporated in the growing chain during a single cycle. As mentioned, the fraction of the overhang filled during a short extension cycle is typically relatively small, e.g., less than about 50% of the length of the overhang.

The number of short extension cycles can be varied, as desired. With each additional cycle performed, the number of template switches increases and therefore the number of crossover points in the resulting chimeric nucleic acids likewise increases.

In the embodiment illustrated in FIG. 1B, after the 20 cycles of short extension are completed, the resulting fragments are subjected to 25 cycles of “assembly PCR” (See reference numeral 115 of the Figure). This assembly PCR is typically performed using conventional shuffling conditions. Most notably, the single strand chain extension produced during the assembly cycles is longer than that produced during the short extension cycles. At the end of the assembly cycling, the distribution of nucleic acid strand lengths approximates that produced using conventional shuffling processes. Additionally, to recover full-length genes, additional cycles may be performed with primers complementary to the end regions of the full-length parent genes. These additional cycles are sometimes referred to as “rescue PCR.” Of note, the depicted procedure does not result in frame shift mutations which would necessarily produce inactive variants.

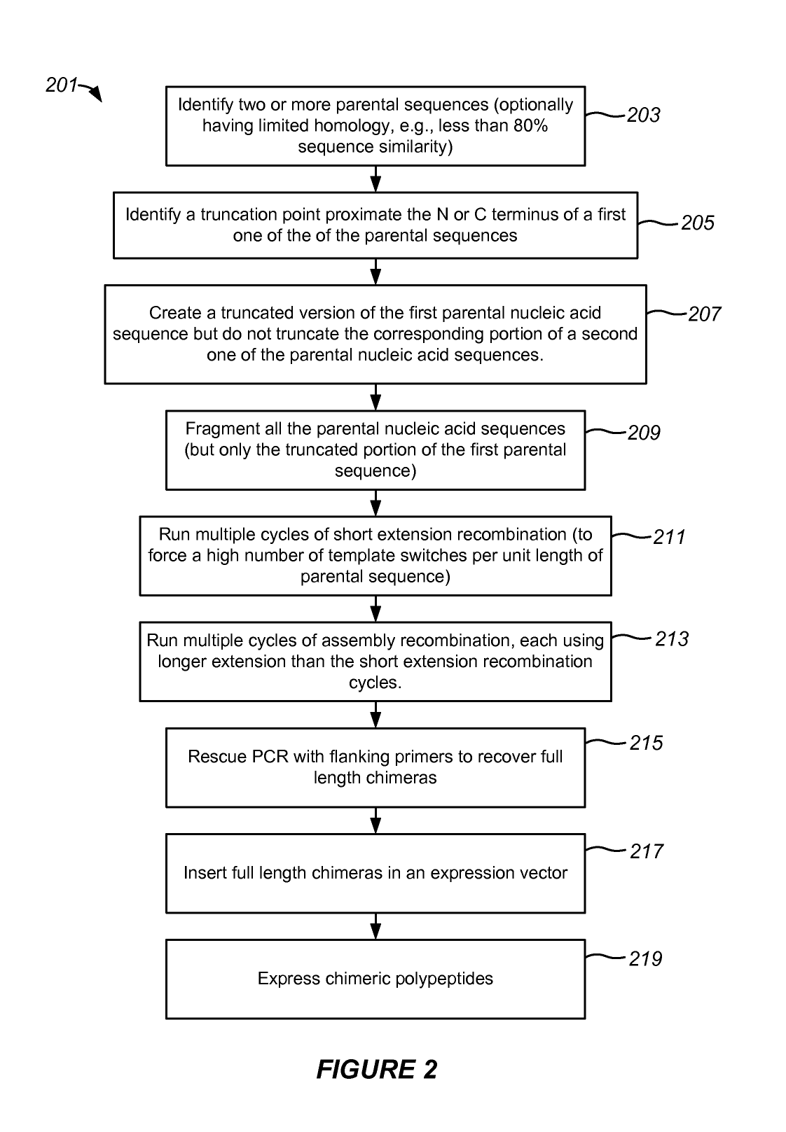

FIG. 2 presents a flowchart depicting an overall shuffling embodiment (201) employing both the truncation procedure depicted in FIG. 1A and the short extension cycling depicted in FIG. 1B. In the process illustrated in this Figure, two or more parental sequences are initially identified for short extension family shuffling, as shown in block 203. The parental sequences under consideration are typically nucleic acid sequences that encode parental proteins of interest. Next, as shown in the depicted sequence, a truncation point is identified in at least one of the parental sequences. While the flowchart identifies the truncation point as being proximate to one of the nitrogen or carbon termini of the parental sequences, this need not be the case. Indeed, in some embodiments, an interior region of the parental sequence is truncated.

The process of identifying the truncation point is depicted by block 205 in FIG. 2. A suitable truncation point is typically one that corresponds to regions of homology between at least two of the parent nucleic acids, particularly between at least one parent that is truncated and at least one other parent that is not truncated at the region of homology. Thus, in some embodiments, the process involves truncating a first parental nucleic acid sequence but not a corresponding portion of a second parental nucleic acid sequence, as indicated in block 207. In some embodiments, the second parental nucleic acid sequence is truncated at a different location, although this need not be the case. It is to be understood that this Figure is for illustration purposes only. It is not intended that the present invention be limited to the use of two parental nucleic acid sequences. Indeed, the present invention finds use with any number of additional parental nucleic acid sequences, as desired.

Next, as indicated in block 209 of FIG. 2, the parental nucleic acids are fragmented to produce a collection of nucleic acid fragments. Fragmentation of the first parental sequence produces fragments that correspond only to a portion of its full-length, as the region that has been excised by the truncation will not be represented in the produced fragments.

In certain embodiments, one or more chemically or biologically synthesized fragments are provided along with the fragments provided from the parental nucleic acids. This approach may be advantageous used to introduce sequence diversity not found in the parental nucleic acids or to bias the amount of a subsequence found in one or more parental nucleic acid. In some embodiments, a significant fraction of the fragments are chemically synthesized (e.g., at least about 5%, or at least about 10%, or at least about 25%, or at least about 50%).

The fragments produced as illustrated in block 209 are combined and then subjected to multiple short extension recombination cycles (e.g., primerless PCR cycles), as illustrated in block 211. In this flowchart, the cycles are conducted in such a manner that only a short extension of the growing strands is accomplished during each cycle. As explained above, in the discussion of FIG. 1B, this forces a relatively high number of template switches per unit length of the parental sequences.

After a sufficient number of short extension recombination cycles are performed, one or more assembly cycles are performed. Each such assembly cycle results in relatively longer chain extension than that achieved by the short extension recombination cycles, as indicated in block 213.

In some embodiments, after the one or more assembly cycles are completed, a rescue PCR operation is conducted, as indicated in block 215. As indicated above, the rescue operation performed with flanking primers complementary to terminal sequences of the full-length parental genes. The rescue PCR will produce nominally full-length genes, having lengths roughly equivalent to those of the parental genes. Of course, these full-length genes will be chimeric, containing some sequences from each of two or more parents.

In some embodiments, the full-length chimeric sequences produced from the performance of the recombination steps depicted in blocks 211, 213, and 215 are then inserted into an expression vector and expressed. This results in the production of chimeric polypeptides, which comprise the desired variant proteins produced by the methods provided herein and illustrated in blocks 217 and 219.

The process flow chart 201 and the associated description above merely exemplify the invention. Numerous variations fall within the scope of the invention. In one example of a variation from the above-described process, truncation occurs near a homologous region (not within the homologous region). In some embodiments, the fragment size obtained for shuffling procedure can vary among parental sequences. For example, one of the parental sequences is fragmentized into fragments having a size of about 50 to about 100 nucleotides while another parental sequence is fragmentized into fragments of about 150 to about 250 nucleotides.

### II. Definitions

The following discussion is provided as an aid in understanding certain aspects and advantages of the disclosed embodiments. Unless otherwise indicated, the practice of the present invention involves conventional techniques commonly used in molecular biology, protein engineering, microbiology, and fermentation science, which are within the skill of the art. Such techniques are well-known and described in numerous texts and reference works well known to those of skill in the art. All patents, patent applications, articles and publications mentioned herein, both supra and infra, are hereby expressly incorporated herein by reference in their entireties and for the purpose indicated by the context in which they are presented.

Unless defined otherwise herein, all technical and scientific terms used herein have the same meaning as commonly understood by one of ordinary skill in the art to which this invention pertains. Many technical dictionaries are known to those of skill in the art. Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the application as a whole.

Also, as used herein, the singular “a”, “an,” and “the” include the plural references, unless the context clearly indicates otherwise. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein. Furthermore, the headings provided herein are not limitations of the various aspects or embodiments of the invention which can be had by reference to the application as a whole. Accordingly, the terms defined immediately below are more fully defined by reference to the application as a whole. Nonetheless, in order to facilitate understanding of the invention, a number of terms are defined below. Unless otherwise indicated, nucleic acids are written left to right in 5′ to 3′ orientation; amino acid sequences are written left to right in amino to carboxy orientation, respectively. As used herein, the term “comprising” and its cognates are used in their inclusive sense (i.e., equivalent to the term “including” and its corresponding cognates).

The terms “protein,” “polypeptide” and “peptide” are used interchangeably to denote a polymer of at least two amino acids covalently linked by an amide bond, regardless of length or post-translational modification (e.g., glycosylation, phosphorylation, lipidation, myristilation, ubiquitination, etc). The terms include compositions conventionally considered to be fragments of full-length proteins or peptides. Included within this definition are D- and L-amino acids, and mixtures of D- and L-amino acids. The polypeptides described herein are not restricted to the genetically encoded amino acids. Indeed, in addition to the genetically encoded amino acids, the polypeptides described herein may be made up of, either in whole or in part, naturally-occurring and/or synthetic non-encoded amino acids. In some embodiments, a polypeptide is a portion of the full-length ancestral or parental polypeptide, containing amino acid additions or deletions (e.g., gaps) or substitutions as compared to the amino acid sequence of the full-length parental polypeptide, while still retaining functional activity (e.g., catalytic activity).

The terms “polynucleotide” and “nucleic acid”, used interchangeably herein, refer to a polymeric form of nucleotides of any length, either ribonucleotides or deoxyribonucleotides. These terms include, but are not limited to, single-, double- or triple-stranded DNA, genomic DNA, cDNA, RNA, DNA-RNA hybrid, polymers comprising purine and pyrimidine bases, and/or other natural, chemically, biochemically modified, non-natural or derivatized nucleotide bases. The following are non-limiting examples of polynucleotides: genes, gene fragments, chromosomal fragments, ESTs, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. In some embodiments, polynucleotides comprise modified nucleotides, such as methylated nucleotides and nucleotide analogs, uracyl, other sugars and linking groups such as fluororibose and thioate, and/or nucleotide branches. In some alternative embodiments, the sequence of nucleotides is interrupted by non-nucleotide components.

“Native sequence” or “wild type sequence” refers to a polynucleotide or polypeptide isolated from a naturally occurring source. Included within “native sequence” are recombinant forms of a native polypeptide or polynucleotide which have a sequence identical to the native form.

“Recombinant” refers to a polynucleotide synthesized or otherwise manipulated in vitro or in vivo (e.g., “recombinant polynucleotide”), to methods of using recombinant polynucleotides to produce gene products in cells or other biological systems, or to a polypeptide (“recombinant protein”) encoded by a recombinant polynucleotide.

Two nucleic acids are “recombined” when sequences from each of the two nucleic acids are combined in a progeny nucleic acid (e.g., a variant or recombinant). Two sequences are “directly” recombined when both of the nucleic acids are substrates for recombination.

In some embodiments, the term “recombinant” includes reference to a polypeptide, polynucleotide, cell, or vector, that has been modified by the introduction of a heterologous nucleic acid sequence. “Recombinant,” “engineered,” and “non-naturally occurring,” when used with reference to a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (i.e., non-recombinant) form of the cell or express native genes that are otherwise expressed at a different level.

“Host cell” or “recombinant host cell” refers to a cell that comprises at least one recombinant nucleic acid molecule. Thus, for example, in some embodiments, recombinant host cells express genes that are not found within the native (i.e., non-recombinant) form of the cell.

“Mutant,” “variant,” and “variant sequence” as used herein, refer to an amino acid (i.e., polypeptide) or polynucleotide sequence that has been altered by at least one substitution, insertion, cross-over, deletion, and/or other genetic operation. For purposes of the present disclosure, mutants and variants are not limited to a particular method by which they are generated. In some embodiments, a mutant or variant sequence has increased, decreased, or substantially similar activities or properties, in comparison to the parental sequence. In some embodiments, the variant polypeptide comprises one or more amino acid residues that have been mutated, as compared to the amino acid sequence of the wild-type polypeptide (e.g., a parent polypeptide). In some embodiments, one or more amino acid residues of the polypeptide are held constant, are invariant, or are not mutated as compared to a parent polypeptide in the variant polypeptides making up the plurality. In some embodiments, the parent polypeptide is used as the basis for generating variants with improved stability, activity, or other property.

“Parental polypeptide,” “parental polynucleotide,” “parent nucleic acid,” and “parent” are generally used to refer to the wild-type polypeptide, wild-type polynucleotide, or a variant used as a starting point in a diversity generation procedure such as a gene shuffling. In some embodiments, the parent itself is produced via shuffling or other diversity generation procedure. In some embodiments, mutants used in shuffling are directly related to a parent polypeptide. In some embodiments, the parent polypeptide is stable when exposed to extremes of temperature, pH and/or solvent conditions and can serve as the basis for generating variants for shuffling. In some embodiments, the parental polypeptide is not stable to extremes of temperature, pH and/or solvent conditions, and the parental polypeptide is evolved to make a robust parent polypeptide from which variants are generated for shuffling.

A “parent nucleic acid” encodes a parental polypeptide.

“Shuffling” and “gene shuffling” refer to methods for introducing diversity into one or more parent polynucleotides to create variant polynucleotides, by recombining a collection of fragments of the parental polynucleotides through a series of chain extension cycles. In certain embodiments, one or more of the chain extension cycles is self-priming; i.e., performed without the addition of primers other than the fragments themselves. Each cycle involves annealing single stranded fragments through hybridization, subsequent elongation of annealed fragments through chain extension, and denaturing. Over the course of shuffling, a growing nucleic acid strand is typically exposed to multiple different annealing partners in a process sometimes referred to as “template switching.” As used herein, “template switching” refers to the ability to switch one nucleic acid domain from one nucleic acid with a second domain from a second nucleic acid (i.e., the first and second nucleic acids serve as templates in the shuffling procedure).

Template switching frequently produces chimeric sequences, which result from the introduction of crossovers between fragments of different origins. The crossovers are created through template switched recombinations during the multiple cycles of annealing, extension, and denaturing. Thus, shuffling typically leads to production of variant polynucleotide sequences. In some embodiments, the variant sequences comprise, a “library” of variants. In some embodiments of these libraries, the variants contain sequence segments from two or more of parent polynucleotides.

When two or more parental polynucleotides are employed, the individual parental polynucleotides are sufficiently homologous that fragments from different parents hybridize under the annealing conditions employed in the shuffling cycles. In some embodiments, the shuffling permits recombination of parent polynucleotides having relatively limited homology. Often, the individual parent polynucleotides have distinct and/or unique domains and/or other sequence characteristics of interest. When using parent polynucleotides having distinct sequence characteristics, shuffling can produce highly diverse variant polynucleotides.

Various shuffling techniques are known in the art (See e.g., U.S. Pat. Nos. 6,917,882, 7,776,598, 8,029,988, 7,024,312, and 7,795,030, all of which are incorporated herein by reference in their entireties.

A “fragment” is any portion of a sequence of nucleotides or amino acids. Fragments may be produced using any suitable method known in the art, including but not limited to cleaving a polypeptide or polynucleotide sequence. In some embodiments, fragments, are produced by using nucleases that cleave polynucleotides. In some additional embodiments, fragments are generated using chemical and/or biological synthesis techniques. In some embodiments, fragments comprise subsequences of at least one parental sequence, generated using partial chain elongation of complementary nucleic acid(s). It is not intended that the invention be limited to any particular fragment(s) or method for generating fragments.

The term “sequence” is used herein to refer to the order and identity of amino acid residues in a protein (i.e., a protein sequence or protein character string) or to the order and identity of nucleotides in a nucleic acid (i.e., a nucleic acid sequence or nucleic acid character string).

A collection of “fragmented nucleic acids” is a collection of nucleic acid fragments. The term “crossover point” as used herein refers to a position in a sequence at which a portion of the sequence changes, or “crosses over” from one source to another (e.g., a terminus of a subsequence involved in an exchange between parental sequences). A “crossover” oligonucleotide has regions of sequence identity to at least two different members of a selected set of nucleic acids (e.g., two different parent polynucleotides). In some embodiments, the nucleotides are homologous, while in other embodiments they are heterologous or non-homologous.

Nucleic acids are generally considered “homologous” when they possess sufficient sequence similarity to permit direct recombination. In some embodiments, homologous nucleic acids are derived, naturally or artificially, from a common ancestor sequence. During natural evolution, this occurs when two or more descendent sequences diverge from a parent sequence over time, i.e., due to mutation and/or natural selection. Under artificial conditions, divergence is produced either by modification using recombinant techniques or de novo synthesis of a desired nucleic acid sequence. In some embodiments, sequences are chemical modified, while in others, modifications are generated through recombinant means. When there is no explicit knowledge about the ancestry of two nucleic acids, homology is typically inferred by sequence comparison between two sequences (i.e., by using sequence alignments). Where two nucleic acid sequences show sequence similarity over a significant portion their lengths, it is inferred that the two nucleic acids share a common ancestor.

As those of skill in the art know, the precise level of sequence similarity used to establish homology varies, depending on a variety of factors. As indicated, two nucleic acids are generally considered to be “homologous” where they share sufficient sequence identity to allow direct recombination to occur between the two nucleic acid molecules. Typically, regions of close similarity spaced roughly the same distance apart are used to permit recombination to occur. The recombination can be in vitro or in vivo, and in some cases, combined.

It should be appreciated, however, that one non-limiting advantage of the present invention is that the methods described herein facility the recombination of more distantly related nucleic acids than standard recombination techniques permit. In particular, sequences from two nucleic acids that are distantly related, or even unrelated can be recombined using forced and/or high frequency template switching. Indeed, in some certain embodiments, parent nucleic acids have only one or a few in common.

Nucleic acids “hybridize” when they associate, typically in solution. Nucleic acids hybridize due to a variety of well characterized physico-chemical forces, such as hydrogen bonding, solvent exclusion, base stacking and the like.

Two nucleic acids “correspond” when they have the same or complementary sequences, when one nucleic acid is a subsequence of the other, and/or when one sequence is derived, by natural (i.e., natural selection) or artificial manipulation (e.g. recombination), from the other.

Nucleic acids are “elongated” or “extended” when additional nucleotides (or other analogous molecules) are incorporated into the nucleic acid. The additional nucleotides generally follow the sequence of a template. In some embodiments of the present invention, the template is a single strand of nucleic acid overhanging a double stranded portion containing the nucleic acid to be elongated. Most commonly, elongation is performed with a polymerase (e.g., a DNA polymerase). DNA polymerases add sequences at the 3′ termini of nucleic acids. Unless stated otherwise, nucleic acid “elongation” and “extension” encompass extension over any length of an overhang from one base to the entire length of the overhang.

As used herein, “incomplete extension” refers to a chain extension process in which only a fraction of an overhanging single stranded segment is filled in prior to terminating a chain extension process. Incomplete extension occurs in double stranded nucleic acids containing an overhanging single strand which serves as a template for polymerase mediated chain extension. In certain embodiments, double stranded nucleic acids containing the overhanging template for incomplete extension are fragments, rather than full-length parent nucleic acids (e.g., full-length genes).

With double stranded fragments used in incomplete extension, the overhang may be between about 5 and about 250 base pairs (on average in a reaction medium), or about 100 to about 200 base pairs (on average). Of course, this is not a rule, and certain applications may employ double stranded fragments having overhangs outside these ranges. In certain embodiments, the incomplete extension is at most about 50% of the overhang, or at most about 45% of the overhang, or at most about 40% of the overhang, or at most about 35% of the overhang, or at most about 30% of the overhang, or at most about 25% of the overhang, or at most about 20% of the overhang, or at most about 15% of the overhang, or at most about 10% of the overhang.

In some methods, incomplete extension is used during a recombination process such as a shuffling process. In some embodiments, incomplete extension recombination processes are performed in a self-priming manner in which only the fragments prime the incomplete extension. In such embodiments, external primers are not employed.

“Annealing” or “hybridizing” refers to the process of establishing a non-covalent, sequence-specific interaction between two or more complementary strands of nucleic acids into a single hybrid, which in the case of two strands is referred to as a duplex. Oligonucleotides, DNA, or RNA will bind to their complement under normal conditions, so two perfectly complementary strands will bind to each other readily. Due to the different molecular geometries of the nucleotides, any inconsistencies between the two strands will make binding between them less energetically favorable. The hybrids may be dissociated by thermal denaturation, also referred to as melting. Here, the solution of hybrids is heated to break the hydrogen bonds between nucleic bases, after which the two strands separate. Most commonly, the pairs of nucleic bases A=T and G≡C are formed.

As used herein, the term “beneficial property” is intended to refer to a phenotypic or other identifiable feature that confers some benefit to a protein or a composition of matter or process associated with the protein. Examples of beneficial properties include an increase or decrease, when compared to a parent protein, in a variant protein's catalytic properties, binding properties, stability when exposed to extremes of temperature, pH, etc., sensitivity to stimuli, inhibition, and the like. Other beneficial properties may include an altered profile in response to a particular stimulus. Further examples of beneficial properties are set forth below.

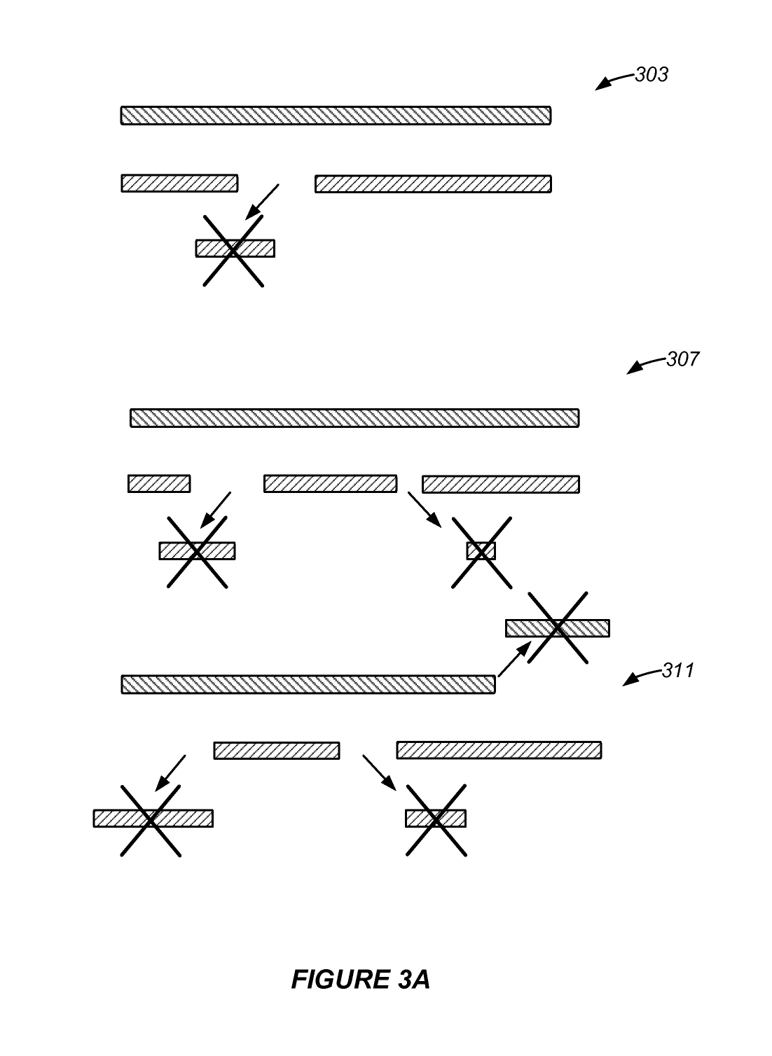

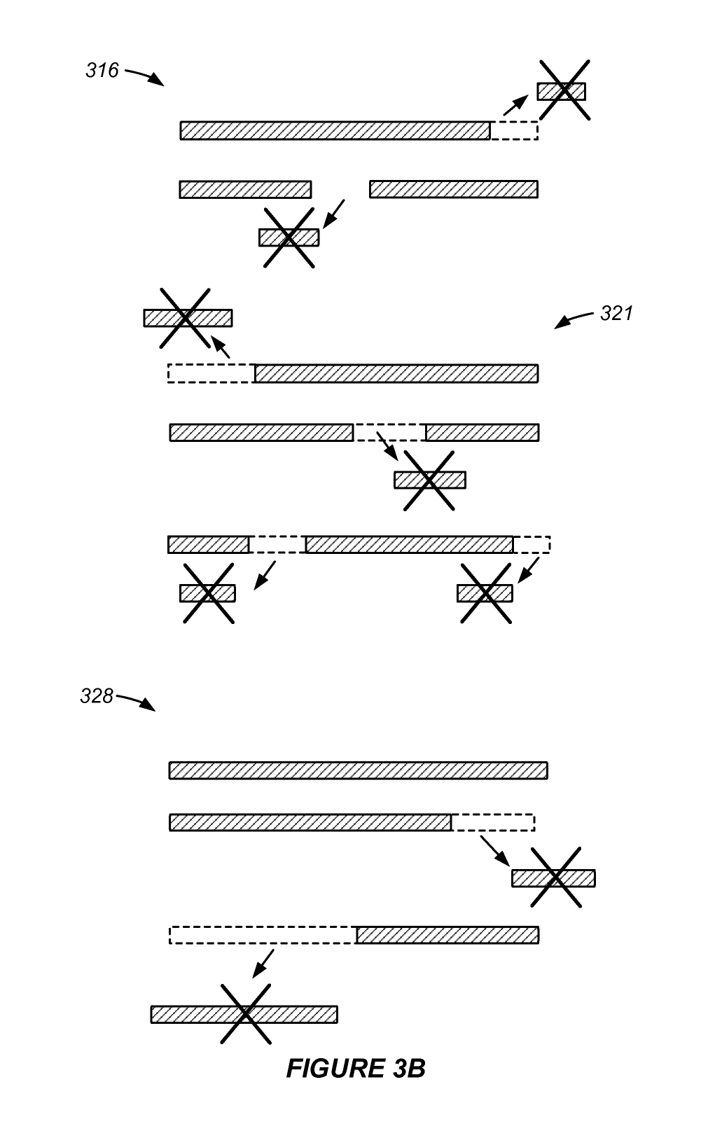

As used herein, the term “truncation point” refers to the sequence location or locations within a full-length parent nucleic acid, such as a full-length gene, where a subsequence of the full-length parent gene is removed. A single truncation point may be used to define a terminal region of the full-length parent nucleic acid to be removed. A pair of truncation points may be used to define an interior region of the full-length parent nucleic acid to be removed. Truncation points may define one, two or more regions of a full-length parent nucleic acid that are to be removed. FIGS. 3A and 3B present a few examples of nucleic acid truncation schemes. In various embodiments, truncation is performed prior to a recombination procedure such as a shuffling procedure.

In some embodiments, the length of the parental nucleotide sequences truncated is between about 15% and about 70% of the full starting length of the parent sequence, or between about 20% and about 50%, or between about 25% and about 40%. In some embodiments, less than about 15% of the full-length of a parent nucleic acid is truncated.

A truncation point may be chosen to facilitate recombination between at least two parent nucleic acids at the truncation point. In one approach to accomplishing this, a region of a first parent nucleic acid is truncated and the corresponding region of a second parent nucleic acid is not truncated. The two parent nucleic acids are then recombined using a technique whereby a recombinant nucleic acid contains a crossover point at the truncation point. To facilitate crossover at the truncation point, the truncation point may be chosen to be within or near a region of high sequence identify between the parent nucleic acid to be truncated and at least one other parent nucleic acid that will not be truncated. In some embodiments, the truncation point is chosen at a region having at least about 80% sequence identity over a length of at least about 15 base pairs. In further embodiments, the truncation point is chosen at a region having at least about 90% sequence identity over a length of at least about 12 base pairs.

Additional or alternative considerations may be applied to identify a truncation point. For example, a truncation point may be chosen to preserve or disrupt a particular domain or other structural region of a parent gene (e.g., an area associated with protein activity such as a catalytic site, or a known secondary structure such as a sheet or a helix, etc.).

A “full-length protein” is a protein having substantially the same sequence as a corresponding protein encoded by a natural gene. The protein can have modified sequences relative to the corresponding naturally encoded gene (e.g., due to recombination and/or selection), but is typically about at least 95% as long as the naturally encoded gene.

A “nucleic acid domain” is a nucleic acid region or subsequence. The domain can be conserved or not conserved between a plurality of homologous nucleic acids. Typically, a domain is delineated by comparison between two or more sequences, i.e., a region of sequence diversity between sequences is a “sequence diversity domain,” while a region of similarity is a “sequence similarity domain.”

An “amplicon” is a nucleic acid made using an amplification reaction such as the polymerase chain reaction (PCR). Typically, the nucleic acid is a copy of a selected nucleic acid. A “primer” is a nucleic acid which hybridizes to a template nucleic acid and permits chain elongation using a polymerase (e.g., a thermostable polymerase such as Taq) under appropriate reaction conditions.

A “library of oligonucleotides” is a set of oligonucleotides. The set can be pooled, or can be individually accessible. Oligonucleotides can be DNA, RNA or combinations of RNA and DNA (e.g., chimeraplasts). In certain embodiments, the library contains a number variant or chimeric nucleic acids produced by a shuffling procedure.

As used herein, the term “cellulase” refers to a category of enzymes capable of hydrolyzing cellulose (β-1,4-glucan or β-D-glucosidic linkages) to shorter cellulose chains, oligosaccharides, cellobiose and/or glucose. In some embodiments, the term “cellulase” encompasses beta-glucosidases, endoglucanases, cellobiohydrolases, cellobiose dehydrogenases, endoxylanases, beta-xylosidases, arabinofuranosidases, alpha-glucuronidases, acetylxylan esterases, feruloyl esterases, and/or alpha-glucuronyl esterases. In some embodiments, the term “cellulase” encompasses hemicellulose-hydrolyzing enzymes, including but not limited to endoxylanases, beta-xylosidases, arabinofuranosidases, alpha-glucuronidases, acetylxylan esterase, feruloyl esterase, and alpha-glucuronyl esterase. A “cellulase-producing fungal cell” is a fungal cell that expresses and secretes at least one cellulose hydrolyzing enzyme. In some embodiments, the cellulase-producing fungal cells express and secrete a mixture of cellulose hydrolyzing enzymes. “Cellulolytic,” “cellulose hydrolyzing,” “cellulose degrading,” and similar terms refer to enzymes such as endoglucanases and cellobiohydrolases (the latter are also referred to as “exoglucanases”) that act synergistically to break down the cellulose to soluble di- or oligosaccharides such as cellobiose, which are then further hydrolyzed to glucose by beta-glucosidase. In some embodiments, the cellulase is a recombinant cellulase selected from β-glucosidases (BGLs), Type 1 cellobiohydrolases (CBH1s), Type 2 cellobiohydrolases (CBH2s), glycoside hydrolase 61s (GH61s), and/or endoglucanases (EGs). In some embodiments, the cellulase is a recombinant Myceliophthora cellulase selected from 13-glucosidases (BGLs), Type 1 cellobiohydrolases (CBH1s), Type 2 cellobiohydrolases (CBH2s), glycoside hydrolase 61s (GH61s), and/or endoglucanases (EGs). In some additional embodiments, the cellulase is a recombinant cellulase selected from EG1b, EG2, EG3, EG4, EG5, EG6, CBH1a, CBH1b, CBH2a, CBH2b, GH61a, and/or BGL.

### III. Process Implementation

### Identifying Parent Nucleic Acids

Initially, a set of parent nucleic acids must be identified or selected for the shuffling procedure. At least two parents are used for shuffling. Frequently more than two parents will be used. For example, 3, 4, 5, 6, 7, 8, 9, 10, 15, 20 or more parents may be used.

In some embodiments, a single “starting” (which may be an “ancestor” sequence) may be employed for purposes of defining a group of two more sequences to be used as “parents” for use in the shuffling process. The starting sequence may be subject to computational or physical mutations to identify or create the parent sequences. Alternatively, no starting sequence is employed, but instead multiple related genes or other nucleic acids are selected as the parent sequences. In some embodiments, at least one of the parents is a wild-type sequence.

In some embodiments where a starting or ancestor sequence is used, mutations are introduced into the starting sequence to create the parent polynucleotides. Such mutations may have been (a) previously identified in the literature as affecting substrate specificity, selectivity, stability, or other beneficial property and/or (b) computationally predicted to improve protein folding patterns (e.g., packing the interior residues of a protein), ligand binding, subunit interactions, family shuffling between multiple diverse homologs, etc. Alternatively, the mutations may be physically introduced into the starting sequence and the expression products screened for beneficial properties. Those sequences having beneficial properties may be used as parent sequences for shuffling. Site directed mutagenesis is one example of a useful technique for introducing mutations, although any suitable method finds use. Thus, alternatively or in addition, the mutants may be provided by gene synthesis, saturating random mutagenesis, semi-synthetic combinatorial libraries of residues, directed evolution, recursive sequence recombination (“RSR”) (See e.g., US Patent Application No. 2006/0223143, incorporated by reference herein in its entirety), gene shuffling, error-prone PCR, and/or any other suitable method. One example of a suitable saturation mutagenesis procedure is described in US Published Patent Application No. 20100093560, which is incorporated herein by reference in its entirety.

The starting protein need not have an amino acid sequence identical to the amino acid sequence of the wild type protein. However, in some embodiments, the starting protein is the wild type protein. In some embodiments, the starting protein has been mutated as compared to the wild type protein. In some embodiments, the starting protein is a consensus sequence derived from a group of proteins having a common property, e.g., a family of proteins.

A non-limiting representative list of families or classes of enzymes which may serve as sources of parent sequences includes, but is not limited to the following: oxidoreducatses (E.C. 1); transferases (E.C. 2); hydrolyases (E.C. 3); lyases (E.C. 4); isomerases (E.C. 5) and ligases (E.C. 6). More specific but non-limiting subgroups of oxidoreducatses include dehydrogenases (e.g., alcohol dehydrogenases (carbonyl reductases), xylulose reductases, aldehyde reductases, farnesol dehydrogenase, lactate dehydrogenases, arabinose dehydrogenases, glucose dehyrodgenase, fructose dehydrogenases, xylose reductases and succinate dehyrogenases), oxidases (e.g., glucose oxidases, hexose oxidases, galactose oxidases and laccases), monoamine oxidases, lipoxygenases, peroxidases, aldehyde dehydrogenases, reductases, long-chain acyl-[acyl-carrier-protein] reductases, acyl-CoA dehydrogenases, ene-reductases, synthases (e.g., glutamate synthases), nitrate reductases, mono and di-oxygenases, and catalases. More specific but non-limiting subgroups of transferases include methyl, amidino, and carboxyl transferases, transketolases, transaldolases, acyltransferases, glycosyltransferases, transaminases, transglutaminases and polymerases. More specific but non-limiting subgroups of hydrolases include ester hydrolases, peptidases, glycosylases, amylases, cellulases, hemicellulases, xylanases, chitinases, glucosidases, glucanases, glucoamylases, acylases, galactosidases, pullulanases, phytases, lactases, arabinosidases, nucleosidases, nitrilases, phosphatases, lipases, phospholipases, proteases, ATPases, and dehalogenases. More specific but non-limiting subgroups of lyases include decarboxylases, aldolases, hydratases, dehydratases (e.g., carbonic anhydrases), synthases (e.g., isoprene, pinene and farnesene synthases), pectinases (e.g., pectin lyases) and halohydrin dehydrogenases. More specific, but non-limiting subgroups of isomerases include racemases, epimerases, isomerases (e.g., xylose, arabinose, ribose, glucose, galactose and mannose isomerases), tautomerases, and mutases (e.g. acyl transferring mutases, phosphomutases, and aminomutases. More specific but non-limiting subgroups of ligases include ester synthases. Other families or classes of enzymes which may be used as sources of parent sequences include transaminases, proteases, kinases, and synthases. This list, while illustrating certain specific aspects of the possible enzymes of the disclosure, is not considered exhaustive and does not portray the limitations or circumscribe the scope of the disclosure.

In some cases, the candidate enzymes useful in the methods described herein are capable of catalyzing an enantioselective reaction such as an enantioselective reduction reaction, for example. Such enzymes can be used to make intermediates useful in the synthesis of pharmaceutical compounds for example.

In certain embodiments, sequences of the selected parent nucleic acids are aligned to identify regions of homology between them. Alignment may be used to determine a level of homology or other similarity between potential parental nucleic acids and hence indicate whether shuffling is likely to be successful. Additionally, alignment may be employed to identify universal primers for subsequent operations such as truncation and rescue PCR. As indicated herein, truncation points are points where crossovers are more likely (or favored) to occur. Amplifying parent nucleic acids using primers having sequences complementary to regions of homology at a defined truncation point will effectively produce a truncated version of the parent gene. As indicated herein, in contrast to many currently used shuffling methods, an advantage of the present invention is that the methods allow shuffling of parental nucleic acids that have relatively low levels of overall homology.

Alignment and sequence comparison algorithms are well-known to those of skill in the art. For example, optimal alignment of sequences for comparison can be algorithms including, but not limited to the local homology algorithm of Smith & Waterman (1981) Adv. Appl. Math. 2:482; the homology alignment algorithm of Needleman & Wench (1970) J. Mol. Biol. 48:443; the search for similarity method of Pearson & Lipan (1988) Proc. Natl. Acad. Sci. USA 85:2444; and computerized implementations of these algorithms (e.g., GAP, BESTFIT, FASTA, and TFASTA).

One example of a suitable alignment algorithm is PILEUP. PILEUP creates a multiple sequence alignment from a group of related sequences using progressive, pairwise alignments to show relationship and percent sequence identity. It also plots a tree or endogamy showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5:151-153. The program employs a multiple alignment procedure, which begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described in Altschul et al. (1990) J. Mol. Biol. 215:403-410. Software for performing BLAST analyses is publicly available through the National Center for Biotechnology Information (ncbi.nlm.nih.gov/). This algorithm involves first identifying high scoring sequence pairs (“HSPs”) by identifying short words of length “W” in the query sequence, which either match or satisfy some positive-valued threshold score “T,” when aligned with a word of the same length in a database sequence. “T” is referred to as the “neighborhood word score threshold” (See, Altschul et al, supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (See Henikoff & Henikoff (1989) Proc. Natl. Acad. Sci. USA 89:10915) alignments (B) of 50, expectation (E) of 10, M=5, N=−4, and a comparison of both strands.

In certain embodiments, at least two of the parent nucleic acids have a sequence identity of about 90% or less. In some embodiments, at least two of the parent nucleic acids have a sequence identity of about 80% or less. In some embodiments, at least two of the parent nucleic acids have a sequence identity of about 70% or less. In some cases, the parent nucleic acids have between about 50 and about 85% sequence identity. In some cases, the parent nucleic acids have between about 55 and about 75% sequence identity. Even lower levels of sequence identify may be possible when the parent nucleic acids have sequence features in common such as motifs. In some embodiments, two or more parent nucleic acids contain identical sequences of as few as about 4 consecutive amino acids (or as few as about 6 consecutive amino acids). Even such low sequence identity can provide a crossover point for the shuffling process. It should also be noted that the truncation process may be employed to delete a low sequence identity region prior to the shuffling procedure.

### Truncation/Excision (Optional)

As explained, one or more of the parent nucleic acids may be identified for optional truncation or excision. An example of truncation is presented in FIG. 1A, which was described above. In some cases, a terminal region of the parent is excised during truncation. N- or C-terminal encoding regions may be excised. These options are depicted in FIG. 1A. Other options are possible and some of these are depicted in FIG. 3A, which shows the excision of one and two interior regions. In option 303, two parental sequences are identified and the lower sequence illustrated in the Figure has a single interior region excised. In some embodiments, which are not explicitly shown in FIG. 3A, the excision occurs prior to fragmentation and shuffling. In option 307, two parental sequences are identified and the lower sequence illustrated in the Figure has two separate interior regions excised. Still further, in option 311, two parental sequences are identified, with the upper sequence illustrated in the Figure having a terminal section excised and the lower sequence illustrated in the Figure having an interior portion and an opposite terminal section excised.

Still other options for truncation are depicted in FIG. 3B. As illustrated in option 316, two parental sequences are truncated, with the upper sequence having a terminal section excised and the lower sequence having only an interior portion excised. In option 321, three parental sequences are identified. The upper sequence has only a terminal region excised. The lower sequence has the opposite terminal region excised, along with an interior region excised. The intermediate sequence has only an interior region excised, with both termini left intact. Finally, in option 328, three parental sequences are identified. The top parental sequence has no regions excised. The intermediate parental sequence has a small terminal region excised. The lower parent sequence has the opposite terminal region excised. The region excised in the lower sequence occupies over one-half the full-length of the parental sequence.

Initially, prior to truncation or excision, a portion or portions of a parent sequence is identified for removal. That is, one or more of the parents that were identified for shuffling are further analyzed to define sequence positions where the truncation or excision is to occur.

In various embodiments, a crossover is forced at the location(s) where the truncation or excision occurs. Therefore, some design consideration may be applied to identify the points or regions where truncation occurs. The degree of sophistication employed to identify these points or regions may vary, depending upon the desired outcome of the method. In certain embodiments, the characteristics of at least one parent polynucleotide, or its corresponding parent polypeptide, are considered in choosing the truncation point. For example, the amount of homology may be taken into consideration when a truncation point is chosen. Typically, though not necessarily, truncation points are more appropriate at positions where two or more parental polynucleotides exhibit relatively high homology levels (e.g., at least about 75 to about 85% sequence identity) or regions near to regions of high homology levels (e.g., truncation points are within about 12 to about 20 base pairs of regions having a high level of sequence identity). This ensures that hybridization and template switching are possible at the truncation point(s). Additionally or alternatively, the truncation points may be chosen to account for the tertiary structure of one or more parental polypeptides. For example, a truncation point may be chosen to preserve (or not unduly disrupt) particular domains, motifs, folds, and the like in a parental polypeptide. In some embodiments, it is desirable to force crossovers at such locations. In certain embodiments, crossovers point, which define truncation/excision points, may be identified using computational techniques such as described in U.S. Pat. No. 7,620,500, which is incorporated herein by reference in its entirety.

In some embodiments, the length of the parental nucleotide sequences truncated is between about 15% to about 70% of the full starting length of the parent sequence. In some embodiments, less than about 15% of the full-length of a parent nucleic acid is truncated. In some cases, such low truncation is desirable when the sequence identity of the regions considered for truncation is low and more than one parent sequences are truncated in the parent sequence pool.

When one parent nucleic acid has a region removed by truncation or excision, at least one other parent nucleic acid should not have its corresponding region truncated. This ensures that there is at least template sequence available adjacent to the crossover point (i.e., the point of excision). Thus, a fragment of a truncated parent having a the truncation point at one end will be able to hybridize to a fragment of a second parent that does not have a corresponding portion removed, and thereby permit chain extension of the first fragment along at least a portion of the corresponding (i.e., non-excised) portion of the second parent. This is depicted in the shuffling schematic shown in FIG. 1A.

Truncation of parent nucleic acids may be accomplished by any suitable technique. One of these, which is described above, is amplification such as PCR amplification using one primer (or set of primers) that is complementary to the terminal sequences of the full-length parent sequence and another primer that is complementary to a truncation point in the interior of the sequence. Excision of an interior portion in a parent sequence can be accomplished by amplification using primers complementary to one or both terminal regions of the full-length parent nucleic acids together with primers that are complementary to the internal regions at the boundaries of the region to be excised. As an alternative to amplification, parent nucleic acids can be modified by cleaving the full-length parents at the truncation points, followed by size separation.

To facilitate subsequent fragmentation of the truncated parent sequences, amplification of the truncated portion may be conducted under conditions that facilitate fragmentation of the amplified product. For example, the amplification may be conducted with nucleotides that, when incorporated in a product nucleic acid, define cleavage sites. Deoxynucleotides containing uracil are one example of such nucleotides. This technique will be described in more detail in the context of the following discussion of fragmentation.

### Mix Parents in Desired Ratios

In some embodiments of the present invention, some or all of the parent nucleic acid fragments are combined in a shuffling medium at the outset, before the short chain extension cycles begin. In some embodiments, the parents are combined before they are fragmented and in some other embodiments they are combined after they are fragmented. The shuffling medium typically includes a water based solution of monomeric nucleotide triphosphates, polymerase, fragments of the parent nucleic acids, and appropriate buffer. Appropriate shuffling media are known in the art and described in various references (See e.g., U.S. Pat. Nos. 6,917,882, 7,776,598, 8,029,988, 7,024,312, 7,795,030, each of which is incorporated herein by reference in its entirety.

In some embodiments, the parent nucleic acids are provided in non-equimolar amounts for assembly. In some other embodiments, all of the parent nucleic acids are provided in equimolar amounts. In embodiments where the parents are present in non-equimolar amounts, the parent(s) present in excess may be chosen based on, for example, one or more properties of the proteins encoded by these parent(s). As a non-limiting example, in one case, multiple parent nucleic acids are identified for shuffling. Of these parents, the polypeptide encoded by one of these parents performs two times better than any of the others. Thus, in some embodiments, the amount of DNA encoding the better-performing parent is added to the shuffling medium prior to assembly significantly exceeds the amount of DNA added from the other parents (i.e., the parents that encode polypeptides that do not perform as well). The shuffling product produced will over-represent the sequences for the better performing parent and hence that parent's sequences will have a higher representation in the final variants. Thus, biasing toward a particular parent or parents provides control over the relative contributions of one or more sequences and/or the mutations present in the over-represented parents. This in turn controls the relative amounts of particular sequences in the final recombination products, e.g., a library of full-length recombinant genes coding the protein(s) of interest.

### Generating Fragments

The parent nucleic acids, which are optionally truncated or excised, are fragmented into fragments of a defined average size or size distribution. In some embodiments, the average length of the fragments is about 50 to about 1500 base pairs. In some embodiments, the average length of the fragments is about 100 to about 1200 base pairs. In some embodiments, the average length of the fragments is about 200 to 800 base pairs. The desired length may be dependent on the average length of the parent nucleic acids. An average fragment size of about 50 to about 300 base pairs may be appropriate for about 1 kb parent sequences. Larger average fragment sizes may be appropriate for longer parent sequences. For example, an average fragment size of about 100 to about 800 base pairs may be appropriate for about 2 kb parent sequences. Further, an average fragment size of about 200 to about 1200 base pairs may be appropriate for about 3 kb parent sequences.

Fragmenting the isolated nucleic acid sequences may be accomplished by any suitable technique, including but not limited to various enzymatic techniques such as DNAse based techniques (e.g., endonuclease cleaving.) and related techniques (See e.g., Stemmer (1994) Rapid evolution of a protein in vitro by DNA shuffling; Nature, 370, 389-391; U.S. Pat. Nos. 5,605,793, 5,830,721, and 5,811,238, each of which is incorporated herein by reference in its entirety) and uracil-based fragmentation (See e.g., U.S. Pat. No. 6,436,675 and Miyazaki (2002); Random DNA fragmentation with endonuclease V: application to DNA shuffling, Nucleic Acids Res. 2002 December 15; 30(24): e139, both of which are incorporated herein by reference). Further, as suggested above, fragments may be produced by introducing uracil into an amplified DNA sequence and then cleaving the amplified sequences at the positions with the introduced uracils.

In the latter embodiment, fragments are produced by first introducing uracil into a DNA sequence during amplification of that sequence, and thereafter cleaving the amplified sequences at the positions with the introduced uracils. In one example, a parent gene (or a truncated portion thereof) is PCR amplified while randomly incorporating dUTP (deoxyuracil triphosphate) in place of where dTTP (deoxythymidinetriphosphate) would normally occur. Some or all of the dTTP may be replaced using these methods. Uracil N-glycosylase and endonuclease IV are used to fragment this PCR product by excision of uracil bases and phosphodiester bond cleavage at these sites, respectively. Some or all of the dTTP may be replaced using these methods. The amount of dTTP replaced depends on the degree of fragmentation achieved. The amplified region sequences, which incorporate uracil, are then fragmented by digestion (e.g., using HK-Ung Thermolabile Uracil N-glycosylase and Endonuclease IV from Epicentre).

Various dTTP and dUTP ratios can be used to determine the desired degree of fragmentation. In various implementations, between about 1 through about 6 mM dUTP concentrations are used. Exemplary mixtures include, but are not limited to the following:

| Volume for: | 1 mM dUTP | 3 mM dUTP | 5 mM dUTP |
|---|---|---|---|
| Sterile water | 60 | 60 | 60 |
| 100 mM dGTP | 10 | 10 | 10 |
| 100 mM dCTP | 10 | 10 | 10 |
| 100 mM dATP | 10 | 10 | 10 |
| 100 mM dTTP | 9 | 7 | 5 |
| 100 mM dUTP | 1 | 3 | 5 |

The uracil N-glycosylase excises uracil and leaves a nick, and Endonuclease IV completes the phosphodiester bond cleavage where nicks reside. The resulting fragmented regions are assembled using, e.g., PCR. In some cases, the assembly is performed using the fragments as produced in the uracil N-glycosylase-Endonuclease IV mixture.

### Short Extension Recombination Cycling

Parent fragments are combined with each other to produce a collection of recombined sequences. Assembly conditions are chosen to allow for base-pairing and extension of complementary fragments. Typically, no primers are employed. Each cycle of PCR increases the average length of the generated fragments length. In some embodiments, recombination occurs via initial short extension cycling followed by longer extension cycling (assembly cycling).

In some embodiments of short extension recombination cycling, the fragments are shuffled under conditions such that chain extension is relatively limited. Thus, the chain extension is short and does not extend the newly synthesized single-strand the entire way to the opposite end of the template to which it is hybridized. The length of the short chain extension and the number of number of cycles of the short extension recombination may be varied to provide the desired degree of crossover. The short extension recombination functions to force an increased number of template switches, thus forcing additional crossovers between different parent nucleic acids.

Each short extension recombination cycle includes (i) annealing single stranded fragments from the two or more parent nucleic acids to produce annealed single stranded fragments, (ii) incompletely extending the annealed single stranded fragments to produce incompletely extended fragments, such that, on average across the annealed fragments from the two or more parent nucleic acids, the extension is not more than about 50% of the overhanging single stranded portion existing prior to extension, (iii) denaturing the incompletely extended single stranded fragments, and (iv) repeating the preceding three operations at least about five times. In some embodiments, the annealing and denaturing conditions are similar to those employed in prior shuffling techniques (See e.g., U.S. Pat. Nos. 6,917,882, 7,776,598, 8,029,988, 7,024,312, and 7,795,030, each of which is incorporated by reference in its entirety).

The average fractional extension per cycle may vary depending on the size of the parent nucleic acid, the size of the fragments, the desired frequency of crossovers, and/or other factors. As examples, the average extension of the single stranded hybridized fragments as a fraction of the overhanging single stranded portion may be limited to not more than about 25%, about 30%, about 35%, about 40%, about 45%, about 50%, about 55%, about 60%, about 65%, about 70%, or about 75%. In some embodiments, the average extension of the single stranded hybridized fragments as a fraction of the overhanging single stranded portion is between about 20 and about 50%.

In some examples of short extension recombination cycling, the initial extension cycles are conducted such that the nucleic acid extends by no more than about 350 nucleotides in each cycle. In some other examples, the nucleic acid extends by no more than about 150 to about 250 nucleotides. In various embodiments, short extension recombination cycling is performed in a manner whereby about 2 to about 3 additional crossover points occur per full-length chimeric sequence when short extension cycle is performed for about 10 cycles prior to the assembly process.

Typically, though not necessarily, the extension portion of the short extension recombination cycles is performed at a lower temperature than would be employed in a corresponding PCR procedure. For example, the extension portion of the short extension recombination cycles may be performed under conditions exposing the annealed single stranded fragments to polymerase and nucleotide triphosphates at a temperature of between about 58° C. and about 75° C. and for a duration of between about 5 and about 20 seconds. The exact conditions are chosen to provide incomplete extension as indicated above. In some examples, the annealing operation is conducted at a temperature of between about 38° C. and about 50° C., the extending operation is conducted at a temperature of between about 58° C. and about 75° C. for a duration of about 10 to about 18 seconds; and the denaturing operation is conducted at a temperature of between about 80° C. and about 160° C. for a duration of about 10 to about 50 seconds.

As indicated herein, the anneal, extension, and denature cycle may be performed for the number of times desired (e.g., at least 5 times) to produce variant sequences. Each repetition of the annealing step involves annealing the incompletely extended single stranded fragments from the previous cycle. In some embodiments, the number of short extension cycles is about 5, about 6, about 7, about 8, about 9, about 10, about 15, about 20, about 25, or about 30.

In some embodiments, the parental nucleic acid fragments are initially exposed to a temperature of about 95° C. for about 1 minute. Thereafter, each short extension cycle includes (i) denaturing at about 95° C. for about 30 seconds, (ii) annealing at about 40° C. for about 20 seconds, and (iii) extending at about 72° C. for about 15 seconds using Taq polymerase, Herculase DNA polymerase or other polymerase that extends at a rate of at least about 1000 nucleotides in 1 minute.

### Assembly PCR

After the requisite number of short extension PCR cycles are conducted to affect a desired level of template switching, additional shuffling cycles are conducted under conditions more typical of normal length extension (See e.g., FIG. 1B). However, this aspect is optional, as the entire shuffling procedure may be conducted using short extension cycles.

A goal of the assembly cycling is to assemble chimeric sequences to full-length. However, each cycle of assembly PCR still provides an opportunity to introduce crossover points between fragments.

To the extent that assembly PCR cycles are conducted, their conditions are generally similar to those of conventional shuffling procedures (See e.g., U.S. Pat. Nos. 6,917,882, 7,776,598, 8,029,988, 7,024,312, and 7,795,030, each of which was previously incorporated by reference in its entirety). In general, the process conditions are be similar to those employed for the short extension cycles except that the extension phase is performed for a significantly longer period of time, e.g., at least about 3 times longer, or at least about 4 times longer, or at least about 5 times longer.

In some embodiments of the assembly cycle, the extending phase is conducted at a temperature of between about 58° C. and about 75° C. for a duration of about 18 to about 60 seconds. These extension times are appropriate for an approximately 1 kb parent polynucleotide. In some embodiments, utilizing approximately 2 kb parental polynucleotides, the extension duration is increased (e.g., to about 120 seconds). In certain embodiments, the assembly cycles are performed at least about 5 times, in order to produce the desired variant sequences. In some embodiments, the number of assembly cycles is about 5, about 6, about 7, about 8, about 9, about 10, about 15, about 20, about 25, or about 30.

In a some embodiments, each assembly cycle includes (i) denaturing at about 95° C. for about 30 seconds, (ii) annealing at about 40° C. about 50° C. for about 20 seconds, and (iii) extending at about 68° C. and about 72° C. for about 75 seconds for an approximately 1 kb parent polynucleotide. In some cases, the annealing phase is performed at a gradually increasing temperature, e.g., about +0.1° C. and about +0.5° C. per cycle.

In some embodiments, the annealing temperature is increased in each cycle to reduce the proportion of non-specific binding pairs in the fragment pool. As indicated above, a low annealing temperature during short extension recombination cycling allows an increasing number of crossover points as annealing between fragments having a relatively low degree of homology is possible. However, keeping the annealing temperature low throughout the assembly cycling may cause non-specific annealing, resulting in a low quality of chimeric gene assembly.

### Rescuing Full-Length Genes or Other Nucleic Acids

At the conclusion of the short extension and the assembly cycles, fragments having a wide range of sequence lengths are produced. To recover full-length nucleic acids having termini corresponding to the full-length parent sequences, further PCR cycles may be performed. Typically, this further PCR is conducted with primers that bracket the full-lengths of the parent nucleic acids. Thus, in these methods, primers complementary to the sequences at the termini of the full-length parents are employed in standard PCR methods using conventional PCR conditions. In contrast, short extension and assembly shuffling steps of the present invention are typically conducted in a primerless fashion, so the “endpoints” of the amplified sequences are not well defined. For a discussion of PCR conditions, see K. Mullis, F. Faloona, S. Scharf, R. Saiki, G. Horn, and H. Erlich, Specific Enzymatic Amplification of DNA in vitro: the Polymerase Chain Reaction, Cold Spring Harb Symp Quant Biol 1986. 51: 263-273, which is incorporated herein by reference in its entirety.

### Expression and Screening

Expression—

Expression of recombinant polypeptides produced by shuffling can be accomplished using any suitable technique, as known in the art. In some embodiments, recombinant polypeptide production is accomplished by incorporating a polynucleotide sequence encoding the polypeptide into an appropriate expression vehicle, e.g., a vector which contains the necessary elements for the transcription and translation of the inserted coding sequence, or in the case of an RNA viral vector, the necessary elements for replication and translation. The expression vehicle is then introduced (e.g., transformed) into a suitable target cell which expresses the polypeptide. Depending on the expression system used, the expressed polypeptide is then isolated by procedures well-established in the art. Indeed, such methods are well known to those skilled in the art and are described in numerous standard texts and reference volumes. Any suitable host expression system finds use in the present invention. Indeed, there is a large variety of host-expression vector systems available, including but not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant plasmid or virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; animal cell systems. Cell-free in vitro polypeptide synthesis systems may also be utilized to produce the polypeptides described herein.

Depending on the host/vector system utilized, any of a number of suitable transcription and translation elements, including constitutive and inducible promoters, may be used in the expression vector. For example, when cloning in bacterial systems, inducible promoters such as pL of bacteriophage lambda, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) may be used; when generating cell lines that contain multiple copies of expression product, SV40-, BPV- and EBV-based vectors may be used with an appropriate selectable marker. Indeed, any suitable promoter and/or other expression element finds use in the present invention. It is not intended that the present invention be limited to any specific promoter(s) and/or other elements.

In embodiments utilizing plant expression vectors, the expression of sequences encoding the polypeptides described herein may be driven by any of a number of promoters. For example, viral promoters such as the 35S RNA and 19S RNA promoters of CaMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680; Broglie et al., 1984, Science 224:838-843) or heat shock promoters, e.g., soybean hsp17.5-E or hsp17.3-B (Gurley et al., 1986, Mol. Cell. Biol. 6:559-565) may be used. (Each of these references is incorporated by reference in its entirety). These constructs can be introduced into plant cells using Ti plasmids, Ri plasmids, plant virus vectors, direct DNA transformation, microinjection, electroporation, etc. Suitable methods are well known to those skilled in the art and are described in well-known texts and reference volumes. In one embodiment an insect expression system that may be used to produce the polypeptides described herein, Autographa californica, nuclear polyhedrosis virus (AcNPV) is used as a vector to express the foreign genes. The virus grows in Spodoptera frugiperda cells. A coding sequence may be cloned into non-essential regions (for example the polyhedron gene) of the virus and placed under control of an AcNPV promoter (for example, the polyhedron promoter). Successful insertion of a coding sequence results in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (See e.g., Smith et al., 1983, J. Virol. 46:584; and U.S. Pat. No. 4,215,051; each of which is incorporated by reference in its entirety)). Additional examples of suitable expression systems are described in reference volumes and texts and are well known in the art.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) results in a recombinant virus that is viable and capable of expressing peptide in infected hosts. (See e.g., Logan & Shenk, 1984, Proc. Natl. Acad. Sci. USA 81:3655-3659). Alternatively, the vaccinia 7.5 K promoter may be used, (See e.g., Mackett et al., 1982, Proc. Natl. Acad. Sci. USA 79:7415-7419; Mackett et al., 1984, J. Virol. 49:857-864; and Panicali et al., 1982, Proc. Natl. Acad. Sci. USA 79:4927-4931; each of which is incorporated by reference in its entirety)).

Non-limiting examples of fungal promoters include, but are not limited to those derived from cellulase genes isolated from a Chrysosporium lucknowense or (i.e., Myceliophthora thermophilia) strain; or a promoter from a T. reesei cellobiohydrolase gene (See e.g., WO2010107303). Other examples of suitable promoters include, but are not limited to promoters obtained from the genes of Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), promoters such as cbh1, cbh2, egl1, egl2, pepA, hfb1, hfb2, xyn1, amy, and glaA (Nunberg et al., 1984, Mol. Cell Biol., 4:2306-2315, Boel et al., 1984, EMBO J. 3:1581-85 and EPA 137280) and mutant, truncated, and hybrid promoters thereof. In a yeast host, useful promoters include, but are not limited to those from the genes for Saccharomyces cerevisiae enolase (eno-1), Saccharomyces cerevisiae galactokinase (gal1), Saccharomyces cerevisiae alcohol dehydrogenase/glyceraldehyde-3-phosphate dehydrogenase (ADH2/GAP), and S. cerevisiae 3-phosphoglycerate kinase. Other useful promoters for yeast host cells include, but are not limited to those described by Romanos et al., 1992, Yeast 8:423-488. In addition, promoters associated with chitinase production in fungi may be used (See e.g., Blaiseau and Lafay, 1992, Gene 120243-248 (filamentous fungus Aphanocladium album; and Limon et al., 1995, Curr. Genet, 28:478-83 (Trichoderma harzianum).

In cell-free polypeptide production systems, components from cellular expression systems are obtained through lysis of cells (eukarya, eubacteria or archaea) and extraction of important transcription, translation and energy-generating components, and/or, addition of recombinant synthesized constituents (See e.g., Shimizu et al. Methods. 2005 July; 36(3):299-304; and Swartz et al. 2004. Methods in Molecular Biology 267:169-182; each of which is incorporated by reference in its entirety)). Thus, cell-free systems can be composed of any combination of extracted or synthesized components to which polynucleotides can be added for transcription and/or translation into polypeptides.

Other expression systems for producing polypeptides described herein will be apparent to those having skill in the art. In some aspects, the present invention provides a plurality of host cell colonies or cultures, wherein each colony or culture expresses one variant and the variants produced by the shuffling procedure described herein.

The polypeptides described herein can be purified by any suitable art-known techniques, including but not limited to reverse phase chromatography, high performance liquid chromatography, ion exchange chromatography, gel electrophoresis, affinity chromatography, and the like. The actual conditions used to purify a particular compound will depend upon the polypeptide(s), and potentially additional factors, including but not limited to net charge, hydrophobicity, hydrophilicity, etc., and will be apparent to those having skill in the art.

Beneficial Properties—

After the genes for the polypeptide variants have been introduced into one or more host cells, the resulting variant proteins having properties of interest are selected. The properties of interest can be any phenotypic or identifiable feature. It is not intended that the present invention be limited to any particular phenotype or identifiable feature.

In some embodiments, a beneficial property or desired activity is an increase or decrease in one or more of the following: substrate specificity, chemoselectivity, regioselectivity, stereoselectivity, stereospecificity, ligand specificity, receptor agonism, receptor antagonism, conversion of a cofactor, oxygen stability, protein expression level, thermoactivity, thermostability, pH activity, pH stability (e.g., at alkaline or acidic pH), inhibition to glucose, and/or resistance to inhibitors (e.g., acetic acid, lectins, tannic acids and phenolic compounds). Other beneficial properties may include an altered profile in response to a particular stimulus; e.g., altered temperature and pH profiles. In some embodiments, the polypeptides encoded by parent nucleic acids and polypeptides encoded by chimeric nucleic acids produced by the methods of this invention act on the same substrate but differ with respect to one or more of the following properties: rate of product formation, percent conversion of a substrate to a product, and/or percent conversion of a cofactor. It is not intended that the present invention be limited to any particular beneficial property and/or desired activity.

In some embodiments, the variants selected following the shuffling methods provided herein are operable over a broad pH range, such as for example, from pH about 2 to pH about 14, from pH about 2 to pH about 12, from pH about 3 to pH about 10, from about pH 5 to about pH 10, pH about 3 to 8, pH about 4 to 7, or pH about 4 to 6.5. In some embodiments, the selected mutants are operable over a broad range of temperatures, such as for example, a range of from about 4° C. to about 100° C., from about 4° C. to about 80° C., from about 4° C. to about 70° C., from about 4° C. to about 60° C., from about 4° C. to about 50° C., from about 25° C. to about 90° C., from about 30° C. to about 80° C., from about 35° C. to about 75° C., or from about 40° C. to about 70° C. In some embodiments, the selected mutants are operable in a solution containing from about 10 to about 50% or more percent organic solvent. Any of the above ranges of operability may be screened as a beneficial property and/or desired activity.

Screening—

Variants may be screened for desired activity using any of a number of suitable techniques. For example, enzyme activity may be detected in the course of detecting, screening for, or characterizing candidate or unknown ligands, as well as inhibitors, activators, and modulators of enzyme activity. Fluorescence, luminescence, mass spectroscopy, radioactivity, and the like may be employed to screen for beneficial properties. Screening may be performed under a range of temperature, pH, and or solvent conditions. Indeed, any suitable screening method known in the art finds use in the present invention. It is not intended that the present invention be limited to any particular screening method and/or reagents.

Various detectable labels may be used in screening. Such labels are moieties that, when attached to, e.g., a polypeptide, renders such a moiety detectable using known detection methods, e.g., spectroscopic, photochemical, electrochemiluminescent, and/or electrophoretic methods. In some embodiments, the label may be a direct label, e.g., a label that is itself detectable or produces a detectable signal, or it may be an indirect label, e.g., a label that is detectable or produces a detectable signal in the presence of another compound. The method of detection will depend upon the label used, and will be apparent to those of skill in the art. Examples of suitable labels include, but are not limited to radiolabels, fluorophores, chromophores, chelating agents, particles, chemiluminescent agents and the like. Such labels allow detection of labeled compounds by a suitable detector, e.g., a fluorometer. Suitable radiolabels include, by way of example and not limitation, include ³H, ¹⁴C, ³²P, ³⁵S, ³⁶Cl, ⁵⁷Co, ¹³¹I and ¹⁸⁶Re.

Fluorescent dyes when conjugated to other molecules or substances generate fluorescence signals that are detectable using standard photodetection systems such as photodetectors employing, e.g., a series of band pass filters and photomultiplier tubes, charged-coupled devices (CCD), spectrographs, etc., as known in the art (See e.g., U.S. Pat. Nos. 4,230,558 and 4,811,218 or in Wheeless et al., 1985, Flow Cytometry: Instrumentation and Data Analysis, pp. 21-76, Academic Press, New York, each incorporated herein by reference in its entirety).

Mass spectrometry encompasses any suitable mass spectrometric format known to those of skill in the art. Such formats include, but are not limited to, Matrix-Assisted Laser Desorption/Ionization, Time-of-Flight (MALDI-TOF), Electrospray (ES), IR-MALDI (See e.g., WO 99/57318 and U.S. Pat. No. 5,118,937, both of which are incorporated herein by reference in its entirety) Ion Cyclotron Resonance (ICR), Fourier Transform and combinations thereof.

“Chromophore” refers to any moiety with absorption characteristics, i.e., moieties that are capable of excitation upon irradiation by any of a variety of photonic sources. Chromophores can be fluorescing or nonfluorescing, and include, but are not limited to dyes, fluorophores, luminescent, chemiluminescent, and electrochemiluminescent molecules.

Examples of suitable indirect labels include enzymes capable of reacting with or interacting with a substrate to produce a detectable signal (e.g., those used in ELISA and EMIT immunoassays), ligands capable of binding a labeled moiety, and the like. Suitable enzymes useful as indirect labels include, by way of example and not limitation, alkaline phosphatase, horseradish peroxidase, lysozyme, glucose-6-phosphate dehydrogenase, lactate dehydrogenase and urease. The use of these enzymes in ELISA and EMIT immunoassays is well known in the art (See e.g., Engvall, 1980, Methods Enzym. 70: 419-439; and U.S. Pat. No. 4,857,453, each of which incorporated herein by reference in its entirety).

Screening generally selects only those variant polypeptides having a desired phenotype or combination of phenotypes. In many embodiments, variants are selected only if they meet or exceed a prespecified threshold level of performance, which typically exceeds the performance level of the parent polypeptide. In some embodiments, however, variants are selected even though they have only the same level of activity as the parent polypeptide. This approach can be useful for generating neutral diversity which may later be useful (e.g., including mutations that are beneficial when taken in combination with other mutations).

### Additional Rounds of Shuffling

In certain embodiments, additional variant sequences are produced by performing the truncation/excision and short extension operations described above using the same parent polynucleotides but employing different truncation or excision patterns. In some embodiments, the truncation and excision patterns are mirror images of those identified in the second step.

Additionally, the library that results from the described shuffling procedures can be used as a source of new parental sequences for subsequent rounds of shuffling. For example, one or more variants that are expressed and identified as having beneficial properties can be selected as parental sequences for a new shuffling procedure as described above.

Further, in various embodiments, shuffling is used in conjunction with a sequence-activity model or other quantitative relationship determination. In some cases, such relationships are used to identify mutations in one or more of the nucleic acid segments. In certain embodiments, such relationships are derived from variant libraries produced by shuffling. Sequence activity relationships so produced may be employed to facilitate further rounds directed evolution, including additional rounds of shuffling. For example, a first set of variants produced by shuffling can be screened to identify at least one polypeptide having enhanced activity for a candidate substrate. The polypeptide(s) so identified from the first recombinant library can then be used as the basis for generating a fine-tuned, higher resolution second plurality for screening the candidate substrate. For example, particularly beneficial mutations appearing in the first library may be used to generate a sequence activity relationship that is then used identify additional mutations. Such mutations may be selected for use in at least one subsequent round of shuffling. The operations of screening and using the results to generate still finer-tuned, still higher resolution pluralities of mutants can be reiterated. In this way, many novel polypeptides with at least one desired activity can be generated and identified. A first plurality can be screened with a novel, unknown or naive substrate or ligand and a second plurality populated with second generation variants generated before testing with the novel, unknown or naive substrate or ligand.

In some embodiments, a sufficient number of variants of the library (e.g., greater than about 10 variants, greater than about 12 variants, greater than about 15 variants, or greater than about 20 variants) exhibit activity on a candidate substrate so that protein sequence activity relationship (ProSAR)-type algorithms may be used to identify important beneficial and/or detrimental mutations among the active variants. The putative more beneficial mutations can then be selected for combination or high weighting in subsequent rounds of region shuffling. ProSAR-type algorithms are described in U.S. Pat. Nos. 7,783,428, 7,747,391, 7,747,393, and 7,751,986, each of which is incorporated herein by reference in its entirety.

### IV. Apparatus

The methods of described herein can be implemented on an appropriately programmed or otherwise configured thermocycler or other nucleic acid amplification apparatus. Indeed, aspects of the invention concern apparatus for preparing chimeric nucleic acids as described herein. Such apparatus may be designed or configured to perform PCR or other amplification procedure under conditions provided to implement short extension recombination cycling as described above and/or truncation/excision of parent nucleic acids as described above. In some embodiments, the apparatus includes a fragmentation module operably coupled to an amplification apparatus.

Any suitable amplification hardware having provisions for receiving, containing, and manipulating PCR media of appropriate compositions may be used. One example of such apparatus is the Biometra T3000 Thermal Cycler, Bio-Rad S1000™ Thermal Cycler. However, the apparatus will additionally include appropriate instructions for implementing methodology as presented herein. The instructions may be provided on board the actual cycling apparatus and may take the form of stored program instructions or may be embodied in a hard coded microprocessor. In some embodiments, the apparatus is a system containing the machine for performing the physical manipulations together with a remote source of such instructions, which source is communicatively connected to the machine over a network, which may be local or wide. In some embodiments, the amplification apparatus includes instructions for calculating or receiving the amplification conditions (e.g., an annealing temperature and an extension temperature) for performing the methods described herein.

The apparatus may be designed or configured to receive user input data to set up one or more cycles to be performed by the apparatus. The input data may include one or more parental nucleic acid sequences, a desired primer set, an extension temperature, an extension duration, an annealing temperature, or other specific features which control the reaction of interest. In some embodiments, the apparatus can receive inputs such as the average extension length for short extension recombination cycling or a desired number of template switches. In response to such high-level inputs, the apparatus, calculates appropriate amplification conditions and implements them accordingly.

In some embodiments, the apparatus is configured or designed to perform the following operations in succession: amplify and/or truncate one or more parental nucleic acids, fragment the one or more parental nucleic acids to produce one or more nucleic acid fragments, reassemble the one or more nucleic acid fragment to produce one or more chimeric nucleic acids and/or amplify the one or more chimeric nucleic acids.

In certain embodiments, the apparatus may be designed or configured to perform primerless, short extension recombination cycling, as described above, where the apparatus contains instructions for chain extension cycling that proceeds no more than about 50% of the overhang, on average, during a particular extension cycle or cycles. For example, the apparatus may be designed or programmed such that the temperature and duration of the short extension recombination cycling are conducted in the manner described above. Further, an apparatus may be designed or programmed such that assembly PRC (which may be primerless) is performed after short extension recombination is performed. In such embodiments, the apparatus is designed or configured such that the duration and temperature of the extension phase of the assembly PCR cycles is controlled in a manner as set forth above. Still further, an apparatus may be designed or programmed to rescue full-length genes as described above.

In certain embodiments, an apparatus is designed or configured to perform truncation or excision of parent nucleic acids. Such apparatus may include provisions for supplying primers having sequences appropriate for amplifying only a truncated portion of one or more parent nucleic acids. In one embodiment, the apparatus is designed or configured to perform this amplification in accordance with conventional PCR protocols. Further, an apparatus for performing truncation or excision of parent nucleic acids may be designed or configured to perform the amplification with uracil-containing deoxynucleotides as described above. For example, the apparatus may be configured to calculate an amount of uracil and an amount of thymidine based on a desired fragment size. In some embodiments, an apparatus for performing truncation or excision of parent nucleic acids may include additional instructions for performing one or more subsequent operation such as short extension recombination cycling, assembly PCR, and/or rescue of full-length genes.

### V. Examples

This work was performed to determine whether the disclosed method of gene shuffling could be used to generate chimera protein that improved a property (in this example, activity). It was found that this method works for shuffling sequences with relatively low homology (>45%) at the amino acid level. Recombination of xylose isomerase was tested using five xylose isomerases (Table 1; CP.XI, AD.XI, RF.XI, RF_FD.XI, and PI.XI) as the parental genes. These five parental genes were synthesized using codon optimization for expression in yeast and to increase sequence identity at the DNA level. Codon optimization was accomplished by using a Saccharomyces cerevisiae codon usage table. One of the xylose isomerases was subjected for codon optimization first as a reference gene and then the other parental genes were optimized towards the reference gene, which increased the sequence identity between parent genes at the DNA level. These genes were inserted into p427-TEF (2 μm plasmid) by homologous recombination in yeast. The flanking homologous sequences used for recombination of all xylose isomerases were

(SEQ ID NO: 11)
5′-GCTCATTAGAAAGAAAGCATAGCAATCTAATCTAAGTTTTGGATCCC
AAACAAA,
and
(SEQ ID NO: 12)
5′-ACTTGATAATGAAAACTATAAATCGTAAAGACATAAGAGATCCGCCA
TATGTTA.

All five parental genes were aligned to identify homologous regions, and internal primers were designed (Table 2). The truncation points were selected where the DNA sequences contain a minimum of 15 base pairs of high homology (>90% homology). All truncation points where selected at sequence locations just prior to helix regions of the parent genes.

Two sets of truncated parental genes were amplified using a dNTP mixture containing 2-3 mM dUTP (Rache Applied Science, Indianapolis, Ind.) and Taq DNA polymerase (Qiagen, Valencia, Calif.). Each set of parental genes containing dUTP were fragmented using HKT™-UNG Thermolabile Uracil N-Glycosylase and Endonuclease IV (Epicentre Biotechnologies, Madison, Wis.) at 37° C. for 120 minutes. During fragmentation, DpnI was added to remove template parental gene. The resulting fragments were pooled together at equal concentrations and assembled using self-priming PCR. Herculase DNA Polymerase (Agilent, Santa Clara, Calif.) was used at 5 units per 100 μl of reaction mixture. Primerless assembly PCR was performed in Biometra T3000 Thermal Cycler (Biometra, Germany) using the following 2 sets of amplification cycles:

1. 95° C. for 1 min

2. 95° C. for 30 sec

3. 40° C. for 20 sec

4. 72° C. for 15 sec

5. Repeat steps 2 to 4, 19 times

6. 95° C. for 30 sec

7. 45° C. for 20 sec, +0.2° C./cycle

8. 72° C. for 75 sec

9. Repeat steps 6 to 8, 24 times

The full-length chimeric genes are recovered from the assembly pool by using the flanking nested primer set

(Flanking.F2,
(SEQ ID NO: 13)
5′-GCTCATTAGAAAGAAAGCATAGCAATCTAATCTAAGTTTTGGATCCC
AAACAAA;
Flanking.R2,
(SEQ ID NO: 14))
5′-ACTTGATAATGAAAACTATAAATCGTAAAGACATAAGAGATCCGCCA
TATGTTA

under the following conditions:

1. 95° C. for 1 min

2. 95° C. for 30 sec

3. 55° C. for 30 sec

4. 72° C. for 75 sec

5. Repeat steps 2 to 4, 19 times

6. 72° C. for 3 min

Full-length chimeric genes containing flanking homologous sequences were mixed with linear p427-TEF yeast expression vector carrying the aminoglycoside phosphotransferase gene for selection using G418, and then subjected to recombinational transformation into yeast host cells using Sigma-Aldrich Yeast-1 kit (St. Louis, Mo.). The colonies were grown on YPD agar with G418 (Geneticin™ Gibco BRL Life Technologies, Inc.). 200 full-length chimeric genes containing the sequences of chimeric genes were analyzed to confirm crossovers within each gene.

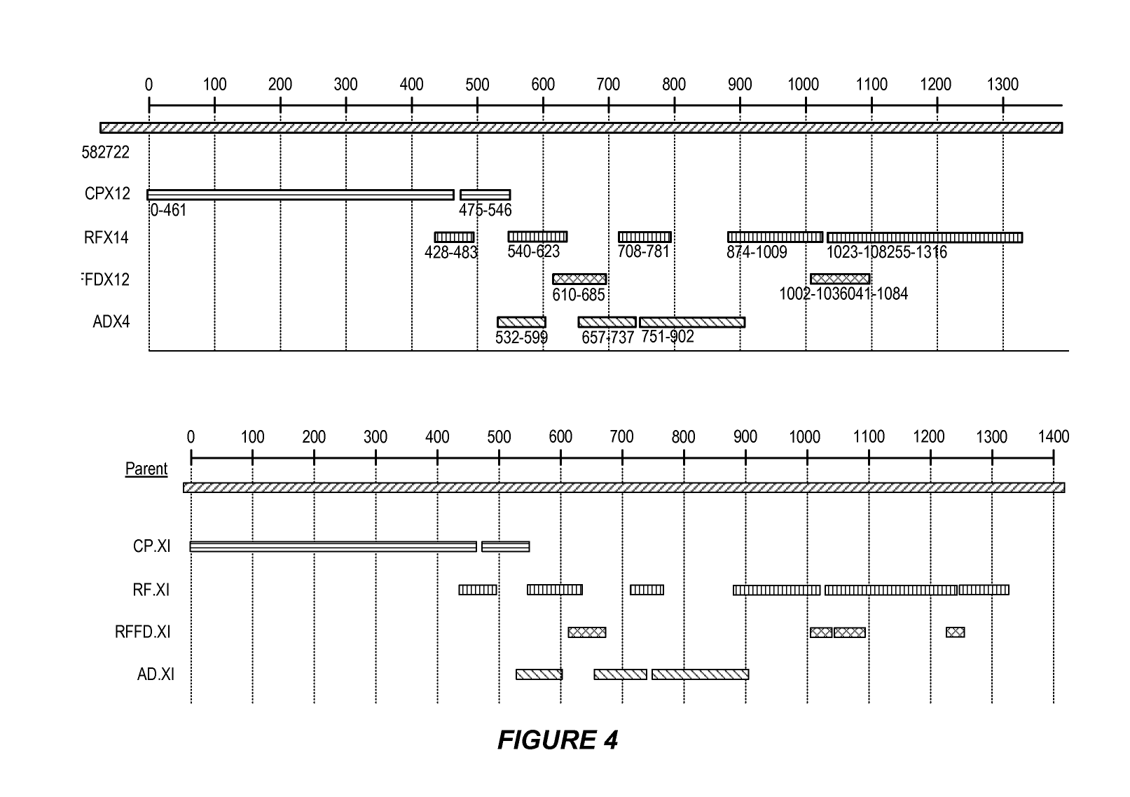

FIG. 4 depicts a full-length chimeric gene selected for improved xylose isomerase activity to provide improved xylose fermentation. The origins (parent genes) of subsequences in this full-length gene are indicated to the left of the bars representing the subsequences making up gene. In FIG. 4, the parent genes are distinguished from one another by their locations at different elevations in the graph (CP.XI.2 on top, RF.XI.4 second from the top, RF.FD.XI.4 third from the top, and AD.XI.4 on the bottom). Numbers under the bars indicate parent fragment range in base pairs.

TABLE 1
Full-length xylose isomerase genes
and their respective SEQ ID Nos

| | Descrip- | | Nucleic Acid | Amino Acid |
|---|---|---|---|---|
| Gene | tion | Species of Origin | SEQ ID NO. | SEQ ID NO |
| CP.XI | xylose | Clostridium | 1 | 2 |
| | isomerase | phytofermentans | | |
| AD.XI | | Abiotrophia | 3 | 4 |
| | | defective | | |
| RF.XI | | Ruminococcus | 5 | 6 |
| | | flavifaciens | | |
| RFFD.XI | | Ruminococcus | 7 | 8 |
| | | flavifaciens FD-1 | | |
| PI.XI | | Phytopthara | 9 | 10 |
| | | infestans | | |

TABLE 2
Primer sequences

| | Par- | | | SEQ |
|---|---|---|---|---|
| Li- | ental | | | ID |
| brary | Gene | Name | Sequence | NO: |
| Set 1 | CP.XI | Flanking.F1 | acggtcttcaatttctc | 15 |
| | | | aagtttcag | |
| | | XI2.R2 | tcgtactggtgcttagt | 16 |
| | | | aggttccttgg | |
| | AD.XI | Flanking.F1 | acggtcttcaatttctc | 17 |
| | | | aagtttcag | |
| | | ADXI4.R4 | gcgaaagaatccatacc | 18 |
| | | | aagaatg | |
| | RF.XI | XI2.F1 | ttatgtcttttggggtg | 19 |
| | | | gtagagaagg | |
| | | Flanking R1 | tattcgtgaaacttcga | 20 |
| | | | acactgtc | |
| | RFFD.XI | RFFDXI2-F1 | tgtcttctggggtggta | 21 |
| | | | gagaagg | |
| | | Flanking R1 | tattcgtgaaacttcga | 22 |
| | | | acactgtc | |
| | PI.XI | PIXI4-F1 | tgtcttttggggtggta | 23 |
| | | | gagaagg | |
| | | PIXI4.R4 | gcgaaacaatccatact | 24 |
| | | | accaatg | |
| set 2 | CP.XI | XI2.F1 | ttatgtcttttggggtg | 25 |
| | | | gtagagaagg | |
| | | Flanking R1 | tattcgtgaaacttcga | 26 |
| | | | acactgtc | |
| | AD.XI | XI2.F1 | ttatgtcttttggggtg | 27 |
| | | | gtagagaagg | |
| | | Flanking R1 | tattcgtgaaacttcga | 28 |
| | | | acactgtc | |
| | RF.XI | Flanking.F1 | acggtcttcaatttctc | 29 |
| | | | aagtttcag | |
| | | RF- | gcgaaagcatccatacc | 30 |
| | | RFFDXI2.R4 | agcaatg | |
| | RFFD.XI | Flanking.F1 | acggtcttcaatttctc | 31 |
| | | | aagtttcag | |
| | | RF- | gcgaaagcatccatacc | 32 |
| | | RFFDXI2.R4 | agcaatg | |
| | PI.XI | PIXI4-F1 | tgtcttttggggtggta | 33 |
| | | | gagaagg | |
| | | PIXI4.R4 | gcgaaacaatccatact | 34 |
| | | | accaatg | |

### DNA Sequence of the Parent Genes

>CP.XI.2
SEQ ID NO: 1
ATGAAGAACTATTTCCCCAACGTCCCAGAAGTCAAATACGAAGGTCCAAA
CTCCACAAATCCTTTCGCTTTTAAATATTATGATGCTAATAAAGTAGTCG
CCGGTAAGACCATGAAGGAGCATTGTAGATTCGCTCTATCCTGGTGGCAC
ACTTTGTGTGCCGGTGGTGCTGATCCATTCGGAGTAACTACTATGGACAG
GACCTACGGTAACATTACCGACCCAATGGAACTAGCTAAGGCCAAAGTTG
ATGCTGGTTTCGAACTGATGACTAAGCTGGGCATCGAGTTCTTCTGCTTC
CATGATGCCGACATTGCTCCAGAAGGTGACACCTTCGAAGAGTCCAAGAA
GAATCTGTTCGAGATTGTTGATTACATCAAGGAGAAGATGGACCAAACCG
GCATCAAGTTGTTATGGGGCACTGCTAACAACTTTAGTCACCCCAGGTTC
ATGCACGGTGCATCAACTTCTTGTAATGCCGATGTTTTCGCTTATGCTGC
TGCGAAAATAAAGAACGCTTTAGATGCGACCATCAAGTTGGGCGGTAAGG
GTTATGTCTTTTGGGGTGGTAGAGAAGGTTACGAGACCCTGCTGAATACT
GACCTGGGCTTAGAACTGGACAACATGGCTAGGCTAATGAAGATGGCCGT
AGAATACGGTAGGGCTAATGGATTCGACGGTGACTTCTACATCGAGCCTA
AACCCAAGGAACCTACTAAGCACCAGTACGACTTCGACACTGCTACCGTA
TTAGCTTTTTTAAGGAAGTACGGGTTGGAAAAAGACTTCAAGATGAACAT
CGAAGCCAATCACGCCACACTAGCAGGCCACACATTCGAGCATGAGTTAG
CTATGGCTAGGGTAAACGGTGCATTCGGTTCTGTTGATGCTAACCAAGGT
GACCCAAACTTAGGATGGGACACGGATCAATTCCCCACAGACGTTCATTC
TGCTACTCTTGCTATGCTGGAGGTCTTGAAAGCCGGTGGTTTCACAAATG
GCGGCCTGAACTTTGATGCGAAAGTTCGTAGGGGTTCATTCGAGTTTGAC
GATATTGCCTATGGTTACATTGCTGGTATGGATACTTTCGCGTTAGGGTT
AATTAAAGCTGCTGAAATCATTGATGACGGTAGAATTGCCAAGTTTGTGG
ATGACAGGTATGCCTCTTACAAGACCGGTATTGGTAAAGCGATCGTTGAC
GGAACTACCTCTTTGGAAGAATTGGAACAATACGTGTTGACTCATTCTGA
ACCTGTCATGCAATCTGGTAGACAAGAGGTTCTGGAAACTATTGTCAACA
ACATATTGTTTAGATAA
>AD.XI4
SEQ ID NO: 3
ATGAGTGAATTGTTCCAAAACATCCCAAAAATCAAATACGAAGGTGCAAA
TTCCAAAAATCCTTTGGCTTTTCATTATTATGATGCTGAAAAAATAGTCC
TCGGTAAGACCATGAAGGAGCATTTGCCATTCGCTATGGCATGGTGGCAC
AATTTGTGTGCCGCTGGTACTGATATGTTCGGACGTGATACTGCGGACAA
GTCCTTTGGTTTGGAAAAAGGCTCAATGGAACATGCTAAGGCCAAAGTTG
ATGCTGGTTTCGAATTTATGGAAAAGCTGGGCATTAAATACTTCTGCTTC
CATGATGTAGACCTTGTTCCAGAAGCTTGCGACATTAAAGAGACCAATTC
TCGACTGGACGAAATTTCTGATTACATCTTGGAGAAGATGAAGGGCACTG
ATATTAAGTGTTTATGGGGCACTGCTAATATGTTTTCTAACCCCAGGTTC
GTGAACGGTGCAGGATCTACTAATAGTGCCGATGTTTACTGTTTTGCTGC
TGCGCAAATAAAGAAAGCATTAGATATTACCGTCAAGTTGGGCGGTAGAG
GTTATGTCTTTTGGGGTGGTAGAGAAGGTTACGAGACCCTGCTGAATACT
GACGTGAAATTTGAACAGGAAAACATTGCTAATCTAATGAAGATGGCCGT
AGAATACGGTAGGTCTATTGGATTCAAAGGTGACTTCTACATCGAGCCTA
AACCCAAGGAACCTATGAAGCACCAGTACGACTTCGACGCTGCTACCGCA
ATAGGTTTTTTAAGGCAGTACGGGTTGGATAAAGACTTCAAATTGAACAT
CGAAGCCAATCACGCCACACTAGCAGGACACTCATTCCAGCATGAGTTAC
GTATTTCTAGTATTAACGGTATGTTGGGTTCTGTTGATGCTAACCAAGGT
GACATGTTGTTAGGATGGGACACGGATGAATTTCCCTTTGACGTTTATGA
TACTACTATGTGTATGTATGAGGTCCTTAAAAACGGTGGTTTGACAGGCG
GCTTTAACTTTGATGCGAAAAATCGTAGGCCTTCATACACGTATGAAGAT
ATGTTCTATGGTTTCATTCTTGGTATGGATTCTTTCGCGTTAGGGTTGAT
AAAAGCTGCTAAATTGATTGAAGAAGGTACACTTGACAATTTTATTAAGG
AAAGGTATAAATCTTTTGAATCCGAAATTGGTAAAAAAATTAGATCCAAA
TCAGCCTCTTTGCAAGAATTGGCAGCTTATGCTGAGGAAATGGGTGCTCC
CGCGATGCCGGGTTCAGGTAGGCAAGAGTATCTGCAAGCTGCTCTCAACC
AAAATTTGTTTGGTGAAGTGTAATAA
>RF.XI.4
SEQ ID NO: 5
ATGGAATTTTTCTCCAACATCGGAAAAATCCAATACCAAGGTCCAAAATC
CACAGATCCTTTGTCTTTTAAATATTATAATCCTGAAGAAGTAATCAACG
GTAAGACCATGAGGGAGCATTTGAAATTCGCTCTATCCTGGTGGCACACT
ATGGGTGGCGATGGTACTGATATGTTCGGATGTGGTACTACGGACAAGAC
CTGGGGTCAATCCGACCCAGCGGCAAGAGCTAAGGCCAAAGTTGATGCTG
CTTTCGAAATTATGGATAAGCTGAGCATTGATTACTACTGCTTCCATGAT
AGAGACCTTTCTCCAGAATATGGCTCCTTGAAAGCGACCAATGATCAACT
GGACATTGTTACTGATTACATCAAGGAGAAGCAGGGCGATAAATTCAAGT
GTTTATGGGGCACTGCTAAATGCTTTGATCACCCCAGGTTCATGCACGGT
GCAGGAACTTCTCCTAGTGCCGATGTTTTCGCTTTTTCTGCTGCGCAAAT
AAAGAAAGCATTAGAATCTACCGTCAAGTTGGGCGGTAATGGTTATGTCT
TTTGGGGTGGTAGAGAAGGTTACGAGACCCTGCTGAATACTAACATGGGC
TTAGAACTGGACAACATGGCTAGGCTAATGAAGATGGCCGTAGAATACGG
TAGGTCTATTGGATTCAAAGGTGACTTCTACATCGAGCCTAAACCCAAGG
AACCTACTAAGCACCAGTACGACTTCGACACTGCTACCGTATTAGGTTTT
TTAAGGAAGTACGGGTTGGATAAAGACTTCAAGATGAACATCGAAGCCAA
TCACGCCACACTAGCACAACACACATTCCAGCATGAGTTACGTGTGGCTA
GGGATAACGGTGTATTCGGTTCTATTGATGCTAACCAAGGTGACGTATTG
TTAGGATGGGACACGGATCAATTCCCCACAAACATTTATGATACTACTAT
GTGTATGTATGAGGTCATTAAAGCCGGTGGTTTCACAAATGGCGGCCTGA
ACTTTGATGCGAAAGCTCGTAGGGGTTCATTCACGCCTGAAGATATTTTC
TATAGTTACATTGCTGGTATGGATGCTTTCGCGTTAGGGTTTAGAGCAGC
TCTTAAATTGATTGAAGACGGTAGAATTGACAAGTTTGTGGCTGACAGGT
ATGCCTCTTGGAATACCGGTATTGGTGCAGATATTATTGCCGGAAAAGCC
GATTTTGCATCATTGGAAAAATATGCTTTGGAAAAAGGTGAAGTTACCGC
GTCATTGTCTTCTGGTAGACAAGAGATGCTGGAATCTATTGTCAACAACG
TATTGTTTAGTTTGTAA
>RF.FD.XI.2
SEQ ID NO: 7
ATGGAATTTTTCAAGAACATCTCTAAGATACCATACGAAGGCAAAGACTC
TACCAATCCATTAGCATTCAAGTACTACAATCCTGACGAAGTAATCGACG
GTAAGAAGATGAGAGACATCATGAAGTTTGCTTTGTCTTGGTGGCATACT
ATGGGAGGTGATGGTACTGATATGTTTGGCTGTGGTACTGCTGATAAGAC
ATGGGGCGAGAATGATCCAGCTGCTAGAGCTAAAGCTAAAGTTGATGCCG
CATTTGAAATCATGCAGAAGTTATCCATTGATTACTTCTGCTTCCATGAT
AGAGATTTGTCTCCAGAGTACGGTTCTTTGAAGGACACAAACGCTCAATT
GGACATTGTCACTGACTACATCAAGGCTAAACAAGCTGAAACCGGTTTGA
AATGTCTTTGGGGTACTGCTAAGTGCTTCGACCATCCAAGATTCATGCAC
GGTGCTGGTACTTCTCCTTCAGCGGATGTCTTCGCATTCTCAGCTGCTCA
AATCAAGAAAGCTCTGGAATCTACCGTCAAGTTGGGTGGAACTGGTTATG
TCTTCTGGGGTGGTAGAGAAGGATATGAAACGTTGTTGAATACTAACATG
GGACTTGAATTGGACAACATGGCTAGGTTGATGAAGATGGCCGTTGAGTA
TGGTAGGTCTATTGGTTTCAAAGGTGACTTCTACATTGAACCTAAGCCAA
AGGAACCAACTAAGCATCAATACGACTTTGACACTGCTACAGTCTTGGGC
TTTCTGAGAAAGTACGGCCTGGACAAAGACTTCAAGATGAACATAGAAGC
CAATCATGCAACTTTAGCGCAACATACCTTCCAGCACGAATTGTGTGTCG
CCAGAACTAATGGTGCTTTCGGTTCTATTGATGCTAATCAAGGTGATCCC
TTGTTGGGTTGGGATACAGATCAGTTTCCTACAAACATCTATGATACTAC
TATGTGCATGTACGAAGTTATCAAAGCTGGTGGTTTCACTAATGGTGGTC
TTAACTTTGATGCTAAAGCTAGAAGAGGTTCTTTCACTCCAGAAGATATT
TTCTATTCTTACATTGCTGGTATGGATGCTTTCGCTTTAGGTTACAAAGC
TGCTTCTAAGCTAATCGCTGATGGTAGGATTGATAGCTTCATTAGCGATA
GATATGCTTCTTGGTCTGAAGGTATTGGTTTGGACATCATTTCCGGCAAA
GCTGATATGGCGGCTTTAGAGAAGTATGCTTTGGAGAAAGGAGAGGTCAC
TGATTCTATCTCTTCTGGAAGACAGGAACTGTTAGAGTCCATTGTTAACA
ACGTAATCTTCAACCTATAATAA
>PI.XI4
SEQ ID NO: 9
ATGCAACATCAAGTGAAAGAATATTTCCCAAACGTCCCAAAAATCACATT
CGAAGGTCAAAATGCCAAAAGTGTTTTGGCTTATCGTGAATATAATGCTT
CAGAAGTAATCATGGGTAAGACCATGGAGGAGTGGTGTAGATTCGCTGTG
TGTTATTGGCACACTTTTGGTAACTCTGGTTCTGATCCGTTCGGAGGTGA
AACTTATACCAATAGATTGTGGAATGAATCATTGGAAAGAGCTAATATTT
CTTCTAGGGAAAGATTGTTGGAAGCTGCTAAGTGCAAAGCTGATGCTGCT
TTCGAAACTTTTACAAAGCTGGGCGTTAAATACTACACCTTCCATGATGT
AGACCTTATTTCAGAAGGTGCCAACCTTGAAGAGTCCCAATCTCTACTGG
ACGAAATTTCTGATTACTTGTTGGATAAGCAGAATCAAACTGGTGTTAGG
TGTTTATGGGGCACTACTAATTTGTTTGGTCACAGAAGGTTCATGAACGG
TGCATCAACTAATCCTGATATGAAAGTTTTCGCTCATGCTGCTGCGAGAG
TAAAGAAAGCAATGGAAATTACCTTGAAGTTGGGCGGTCAAAATTTTGTC
TTTTGGGGTGGTAGAGAAGGTTTCCAGTCCATTCTGAATACTGACATGAA
AACTGAACTGGATCACATGGCTGCTTTTTTTAAGTTGGTCGTAGCATACA
AAAAGGAACTTGGAGCCACATTTCAATTCTTGGTCGAGCCTAAACCCAGG
GAACCTATGAAGCACCAGTACGACTACGACGCTGCTACCGTAGTAGCTTT
TTTACATACGTACGGGTTGCAAAATGACTTCAAATTGAACATCGAACCCA
ATCACACCACACTAGCAGGACACGATTACGAGCATGATATATATTATGCT
GCTAGTTACAAAATGTTGGGTTCTGTTGATTGTAACACAGGTGACCCGTT
GGTAGGATGGGACACGGATCAATTTTTGATGGACGAAAAAAAAGCTGTTT
TGGTTATGAAAAAGATCGTTGAAATCGGTGGTTTGGCACCAGGCGGCTTG
AACTTTGATGCGAAAGTTCGTAGGGAATCAACCGATTTGGAAGATATTTT
CATTGCTCACATTGGTAGTATGGATTGTTTCGCGAGAGGGTTGAGACAAG
CTGCTAAATTGCTTGAAAAAAATGAACTTGGCGAATTGGTTAAGCAAAGG
TATGCATCTTGGAAATCCACACTTGGTGAAAGAATTGAACAAGGACAAGC
CACTTTGGAAGAAGTGGCAGCTTATGCTAAGGAAAGTGGTGAACCCGATC
ATGTGTCAGGTAAGCAAGAGTTGGCGGAACTTATGTGGAGCACAGTTGCG
TTGGCTACAGGGATTTGGCAAGATCATGTTACTTGTTCTTTGACTAAAAA
TTGGTGTTAA

### Protein Sequence of the Parent Genes

CP.XI.2
SEQ ID NO: 2
MKNYFPNVPEVKYEGPNSTNPFAFKYYDANKVVAGKTMKEHCRFALSWWHTLCAGGADPFGV
TTMDRTYGNITDPMELAKAKVDAGFELMTKLGIEFFCFHDADIAPEGDTFEESKKNLFEIVD
YIKEKMDQTGIKLLWGTANNFSHPRFMHGASTSCNADVFAYAAAKIKNALDATIKLGGKGYV
FWGGREGYETLLNTDLGLELDNMARLMKMAVEYGRANGFDGDFYIEPKPKEPTKHQYDFDTA
TVLAFLRKYGLEKDFKMNIEANHATLAGHTFEHELAMARVNGAFGSVDANQGDPNLGWDTDQ
FPTDVHSATLAMLEVLKAGGFTNGGLNFDAKVRRGSFEFDDIAYGYIAGMDTFALGLIKAAE
IIDDGRIAKFVDDRYASYKTGIGKAIVDGTTSLEELEQYVLTHSEPVMQSGRQEVLETIVNN

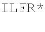

AD.XI.4
SEQ ID NO: 4
MSELFQNIPKIKYEGANSKNPLAFHYYDAEKIVLGKTMKEHLPFAMAWWHNLCAAGTDMFGR
DTADKSFGLEKGSMEHAKAKVDAGFEFMEKLGIKYFCFHDVDLVPEACDIKETNSRLDEISD
YILEKMKGTDIKCLWGTANMFSNPRFVNGAGSTNSADVYCFAAAQIKKALDITVKLGGRGYV
FWGGREGYETLLNTDVKFEQENIANLMKMAVEYGRSIGFKGDFYIEPKPKEPMKHQYDFDAA
TAIGFLRQYGLDKDFKLNIEANHATLAGHSFQHELRISSINGMLGSVDANQGDMLLGWDTDE
FPFDVYDTTMCMYEVLKNGGLTGGFNFDAKNRRPSYTYEDMFYGFILGMDSFALGLIKAAKL
IEEGTLDNFIKERYKSFESEIGKKIRSKSASLQELAAYAEEMGAPAMPGSGRQEYLQAALNQ

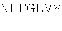

RF.XI.4
SEQ ID NO: 6
MEFFSNIGKIQYQGPKSTDPLSFKYYNPEEVINGKTMREHLKFALSWWHTMGGDGTDMFGCG
TTDKTWGQSDPAARAKAKVDAAFEIMDKLSIDYYCFHDRDLSPEYGSLKATNDQLDIVTDYI
KEKQGDKFKCLWGTAKCFDHPRFMHGAGTSPSADVFAFSAAQIKKALESTVKLGGNGYVFWG
GREGYETLLNTNMGLELDNMARLMKMAVEYGRSIGFKGDFYIEPKPKEPTKHQYDFDTATVL
GFLRKYGLDKDFKMNIEANHATLAQHTFQHELRVARDNGVFGSIDANQGDVLLGWDTDQFPT
NIYDTTMCMYEVIKAGGFTNGGLNFDAKARRGSFTPEDIFYSYIAGMDAFALGFRAALKLIE
DGRIDKFVADRYASWNTGIGADIIAGKADFASLEKYALEKGEVTASLSSGRQEMLESIVNNV

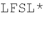

RF.FD.XI.4
SEQ ID NO: 8
MEFFKNISKIPYEGKDSTNPLAFKYYNPDEVIDGKKMRDIMKFALSWWHTMGGDGTDMFGCG
TADKTWGENDPAARAKAKVDAAFEIMQKLSIDYFCFHDRDLSPEYGSLKDTNAQLDIVTDYI
KAKQAETGLKCLWGTAKCFDHPRFMHGAGTSPSADVFAFSAAQIKKALESTVKLGGTGYVFW
GGREGYETLLNTNMGLELDNMARLMKMAVEYGRSIGFKGDFYIEPKPKEPTKHQYDFDTATV
LGFLRKYGLDKDFKMNIEANHATLAQHTFQHELCVARTNGAFGSIDANQGDPLLGWDTDQFP
TNIYDTTMCMYEVIKAGGFTNGGLNFDAKARRGSFTPEDIFYSYIAGMDAFALGYKAASKLI
ADGRIDSFISDRYASWSEGIGLDIISGKADMAALEKYALEKGEVTDSISSGRQELLESIVNN

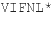

PI.XI.4
SEQ ID NO: 10
MQHQVKEYFPNVPKITFEGQNAKSVLAYREYNASEVIMGKTMEEWCRFAVCYWHTFGNSGSD
PFGGETYTNRLWNESLERANISSRERLLEAAKCKADAAFETFTKLGVKYYTFHDVDLISEGA
NLEESQSLLDEISDYLLDKQNQTGVRCLWGTTNLFGHRRFMNGASTNPDMKVFAHAAARVKK
AMEITLKLGGQNFVFWGGREGFQSILNTDMKTELDHMAAFFKLVVAYKKELGATFQFLVEPK
PREPMKHQYDYDAATVVAFLHTYGLQNDFKLNIEPNHTTLAGHDYEHDIYYAASYKMLGSVD
CNTGDPLVGWDTDQFLMDEKKAVLVMKKIVEIGGLAPGGLNFDAKVRRESTDLEDIFIAHIG
SMDCFARGLRQAAKLLEKNELGELVKQRYASWKSTLGERIEQGQATLEEVAAYAKESGEPDH

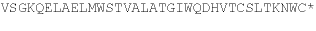

TABLE 3
Sequence similarities of parents used in this method, (a)
protein sequence similarity, (b) DNA sequence similarity.

| | | | | |
|---|---|---|---|---|
| AD.XI.4 | CP.XI.2 | RF.XI.4 | RFFD.XI.2 | PI.XI.4 |

(a)

| | | | | | |
|---|---|---|---|---|---|
| AD.XI.4 | 100 | 64 | 64 | 64 | 44 |
| CP.XI.2 | | 100 | 67 | 67 | 46 |
| RF.XI.4 | | | 100 | 89 | 42 |
| RFFD.XI.2 | | | | 100 | 42 |
| PI.XI.4 | | | | | 100 |

(b)

| | | | | | |
|---|---|---|---|---|---|
| AD.XI.4 | 100 | 78 | 81 | 67 | 68 |
| CP.XI.2 | | 100 | 82 | 68 | 64 |
| RF.XI.4 | | | 100 | 79 | 63 |
| RFFD.XI.2 | | | | 100 | 54 |
| PI.XI.4 | | | | | 100 |

### VI. Other Embodiments

While various specific embodiments have been illustrated and described, it will be appreciated that various changes can be made without departing from the spirit and scope of the invention(s). For example, all the techniques described above may be used in various combinations.

Indeed, while the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications and patent documents cited herein are incorporated herein by reference (for the purposes indicated by their contexts in the specification) as if each such publication or document was specifically and individually indicated to be incorporated herein by reference. Citation of publications and patent documents is not intended as an indication that any such document is pertinent prior art, nor does it constitute any admission as to the contents or date of the same.

What is claimed is:

1. A method of conducting nucleic acid recombination to facilitate incorporation of crossovers in variant sequences, the method comprising:
(a) combining fragments of two or more parent nucleic acids;
(b) annealing single stranded fragments from the two or more parent nucleic acids to produce annealed single stranded fragments, wherein at least some of the annealed single stranded fragments have overhanging single stranded portions attached to a double stranded portion;
(c) incompletely extending the annealed single stranded fragments to produce incompletely extended single stranded fragments, wherein, on average across the annealed fragments from the two or more parent nucleic acids, the extension is not more than about 50% of the overhanging single stranded portion of the annealed single stranded fragments existing prior to extension;
(d) denaturing the incompletely extended single stranded fragments produced in (c); and
(e) repeating (b)-(d) at least about 5 times to produce variant sequences, wherein the repetitions of (b) comprise annealing the incompletely extended single stranded fragments from (c).

2. The method of claim 1, wherein at least one of the two or more parent nucleic acids comprises a wild type nucleic acid sequence.

3. The method of claim 1 or 2, wherein the two or more parent nucleic acids comprise sequences having between about 50 and about 85 percent sequence identity.

4. The method of claim 1, 2, or 3, wherein the fragments of two or more parent nucleic acids are produced by endonuclease cleaving.

5. The method of claim 1, 2, or 3, wherein fragments of two or more parent nucleic acids are produced by cleavage at positions comprising uracil in the parent nucleic acids.

6. The method of any of the foregoing claims, wherein the fragments of the two or more parent nucleic acids are produced by a method that does not include polymerase extension on a template comprising an unfragmented full-length parent nucleic acid.

7. The method of any of the foregoing claims, wherein the fragments are not produced by a method in which fragments are produced by extensions from primers.

8. The method of any of the foregoing claims, further comprising, prior to (a), truncating a region of at least one of the two or more parent nucleic acids to produce a truncated fragment.

9. The method of claim 8, wherein at least one of the two or more parent nucleic acids is not truncated at a region corresponding the region truncated in the at least one parent nucleic acid.

10. The method of any of the foregoing claims, wherein (c) comprises incompletely extending the annealed single stranded fragments by not more than about 35% of the overhanging single stranded portion, on average.

11. The method of any of the foregoing claims, wherein incompletely extending in (c) comprises exposing the annealed single stranded fragments to polymerase and nucleotide triphosphates at a temperature of between about 58° C. and about 75° C. for a duration of between about 5 seconds and about 20 seconds.

12. The method of any of the foregoing claims, wherein (e) comprises repeating (b)-(d) at least about 10 times.

13. The method of any of the foregoing claims, wherein (e) comprises repeating (b)-(d) at least about 15 times.

14. The method of any of the foregoing claims, wherein
the annealing in (b) is conducted at a temperature of between about 38° C. and about 50° C.;
the extending in (c) is conducted at a temperature of between about 58° C. and about 75° C. for a duration of about 10 seconds to about 18 seconds; and
the denaturing in (d) is conducted at a temperature of between about 80° C. and about 160° C. for a duration of about 10 seconds to about 50 seconds.

15. The method of any of the foregoing claims, wherein incompletely extending in (c) comprises a self-priming reaction in a medium that does not contain external primers.

16. The method of any of the foregoing claims, wherein the incompletely extending in (c) is performed in a medium that does not contain unfragmented full-length parent nucleic acids.

17. The method of any of the foregoing claims, further comprising, after (e):
(f) repeating (b);
(g) extending the annealed single stranded fragments to produce extended single stranded fragments, wherein, on average across the annealed fragments from the two or more parent nucleic acids, the extension is significantly greater than the extensions in (c);
(h) denaturing the extended single stranded fragments produced in (g); and
(i) repeating (f)-(h) at least about 10 times.

18. The method of claim 17, wherein
extending the single stranded fragments in (g) is conducted at a temperature of between about 58° C. and about 75° C. for a duration of about 18 seconds to about 60 seconds.

19. The method of claim 18, wherein the annealing temperature is gradually increased during successive repetition recited in (i).

20. The method of any of claims 1-16, further comprising (f) identifying one or more recombinant proteins encoded by one or more variant sequences from (e), wherein the one or more recombinant proteins have at least one beneficial property.

21. The method of claim 20, wherein at least one of the recombinant proteins is an enzyme.

22. The method according to claim 21, wherein at least one enzyme is a cellulase, reductase, transferase, transaminase, isomerase, protease, oxidase, kinase, synthase, or esterase.

23. The method of claim 20, further comprising:
assaying and sequencing the one or more recombinant proteins; and
developing a sequence activity model from assay and sequence information for the recombinant proteins.

24. The method of any of the foregoing claims, further comprising fragmenting the two or more parent nucleic acids.

25. A method of conducting nucleic acid recombination to facilitate incorporation of crossovers in variant sequences, the method comprising:
(a) truncating a region of at least one of two or more parent nucleic acid to produce at least one truncated parent nucleic acid;
(b) fragmenting and combining the at least one truncated parent nucleic acid of (a) and at least one other parent nucleic acid that is not truncated in a region corresponding to the region truncated in the at least one truncated parent nucleic acid;
(c) annealing single stranded fragments from the two or more parent nucleic acids, to produce annealed single stranded fragments, wherein at least some of the annealed single stranded fragments have overhanging single stranded portions attached to a double stranded portion;
(d) incompletely extending the annealed single stranded fragments, to produce incompletely extended single stranded fragments, wherein, on average across the annealed fragments from the two or more parent nucleic acids, the extension is not more than about 50% of the overhanging single stranded portion of the annealed single stranded fragments;
(e) denaturing the incompletely extended single stranded fragments produced in (d); and
(f) repeating (c)-(e) to produce variant sequences, wherein the repetitions of (c) comprise annealing the incompletely extended single stranded fragments from (d).

26. The method of claim 25, wherein (a) comprises truncating at least one of the two or more parent nucleic acids by removing a segment encoding a nitrogen terminal region of a protein encoded by the at least one parent nucleic acid and truncating at least one other of the two or more parent nucleic acids by removing a segment encoding a carbon terminal region of a protein encoded by the other parent nucleic acid.

27. The method of claim 25 or 26, wherein the truncating comprises amplifying the at least one parent nucleic acid in the presence of at least one primer complementary to an internal sequence of the at least one parent nucleic acid to produce at least one amplified parent nucleic acid.

28. The method of claim 27, wherein the amplifying comprises incorporating uracil nucleotides in the amplicons of at least one amplified parent nucleic acid.

29. The method of claim 28, wherein the fragmenting comprises cleaving the amplicons at the uracil containing positions of the amplified parent nucleic acids.

30. The method of any of claims 25-29, wherein (d) comprises incompletely extending the annealed single stranded fragments by not more than about 25% of the overhanging single stranded portion, on average.

31. The method of any of claims 25-30, wherein
the annealing in (c) is conducted at a temperature of between about 38° C. and about 50° C.;
the extending in (d) is conducted at a temperature of between about 58° C. and about 75° C. for a duration of about 10 to about 18 seconds; and
the denaturing in (e) is conducted at a temperature of between about 80° C. and about 160° C. for a duration of about 10 to about 50 seconds.

32. The method of any of claims 25-31, further comprising, after (f):
(g) repeating (c);
(h) extending the annealed single stranded fragments, to produce extended singled stranded fragments, wherein, on average across the annealed fragments from the two or more parent nucleic acids, the extension is significantly greater than the extensions in (d);
(i) denaturing the extended single stranded fragments produced in (h); and
(j) repeating (g)-(i) at least about 10 times.

33. A method of conducting nucleic acid recombination to facilitate incorporation of crossovers in variant sequences, the method comprising:
(a) truncating a region of at least one of two or more parent nucleic acid to produce at least one truncated parent nucleic acid;
(b) fragmenting and combining the at least one truncated parent nucleic acid of (a) and at least one other parent nucleic acid that is not truncated in a region corresponding to the region truncated in the at least one truncated parent nucleic acid;
(c) annealing single stranded fragments from the two or more parent nucleic acids to produce annealed single stranded fragments, wherein at least some of the annealed single stranded fragments have overhanging single stranded portions attached to a double stranded portion;
(d) extending the annealed single stranded fragments to produce extended single stranded fragments;
(e) denaturing the extended single stranded fragments produced in (d); and
(f) repeating (c)-(e) at least about 5 times to produce variant sequences, wherein the repetitions of (c) comprise annealing the extended single stranded fragments from (d).

34. The method of claim 33, wherein (a) comprises truncating at least two of the two or more parent nucleic acids.

35. The method of claim 34, wherein (a) comprises truncating at least one of the two or more parent nucleic acids by removing a segment encoding a nitrogen terminal region of a protein encoded by the at least one parent nucleic acid and truncating at least one other of the parent nucleic acids by removing a segment encoding a carbon terminal region of a protein encoded by the other parent nucleic acid.

36. The method of claim 33, 34, or 35, wherein the truncating comprises amplifying the at least one parent nucleic acid in the presence of at least one primer complementary to an internal sequence of the at least one parent nucleic acid to produce at least one amplified parent nucleic acid.

37. The method of claim 36, wherein the amplifying comprises incorporating uracil nucleotides in the amplicons of at least one amplified parent nucleic acid.

38. The method of claim 37, wherein the fragmenting comprises cleaving the amplicons at the uracil containing positions of the amplified parent nucleic acids.

39. The method of any of claims 33-38, wherein the two or more parent nucleic acids have substantially the same length and have between about 50 and about 85% sequence identity.

40. The method of any of claims 33-38, further comprising aligning the parent nucleic acids to identify one or more regions of homology.

41. The method of claim 40, further comprising creating a primer complementary to at least one identified region of homology, wherein the primer is used in truncating the region of the at least one parent nucleic acid to produce the at least one truncated parent nucleic acid.

42. The method of claim 40, further comprising creating a primer complementary to at least one identified region of homology, wherein the primer is used in recovering full-length nucleic acids from the variant sequences.

43. The method of any of claims 33-42, wherein, in (b), fragments from the two or more parent nucleic acids are combined in non-equimolar amounts.

44. The method of any of claims 33-43, wherein, in (b), fragments from the two or more parent nucleic acids are combined in substantially equimolar amounts.

45. The method of any of claims 33-44, wherein the extending in (d) comprises incompletely extending the single stranded fragments to produce extended single stranded fragments that are incompletely extended, wherein, on average across the annealed fragments from the two or more parent nucleic acids, the extension is not more than about 30% of the overhanging single stranded portion.

46. The method of any of claims 33-45, further comprising extending the variant sequences to produce nucleic acids having substantially the same length as at least one of the parent nucleic acids.

47. The method of claim 46, wherein extending the variant sequences comprises amplifying the variant sequences with flanking primers complementary to the terminal regions of at least one of the parent nucleic acids.

48. The method of any of claims 33-47, wherein at least one of the two or more parental nucleic acids comprises a wild type nucleic acid.

49. The method of any of claims 33-48, wherein (f) comprises repeating (c)-(e) at least about 20 times.

50. The method of any of claims 33-49, further comprising (g) identifying one or more recombinant proteins encoded by one or more variant sequences from (f), wherein the one or more recombinant proteins have at least one beneficial property.

51. The method of claim 50, wherein at least one of the recombinant proteins is an enzyme.

52. The method according to claim 51, wherein at least one of enzyme is a cellulase, reductase, transferase, transaminase, isomerase, protease, oxidase, kinase, synthase, or esterase.

53. The method of any of claims 33-52, wherein the fragmenting comprises a process that does not include polymerase extension on a template comprising an unfragmented full-length or truncated parent nucleic acid.

54. The method of any of claims 33-53, wherein the fragmenting comprises a process in which fragments are not produced by extensions from primers.

55. The method of any of claims 33-54, wherein the extending in (d) comprises a self-priming reaction in a medium that does not contain external primers.

56. The method of any of claims 33-55, wherein the extending in (d) is performed in a medium that does not contain unfragmented full-length parent nucleic acids.
